SEQUENCE LISTING (25 sequences)

SEQ ID NO: 1 - DNA, 24 bp, Artificial Sequence
  Description of Artificial Sequence Synthetic oligonucleotide
tgctgcttttgtgcttttgtgctt

SEQ ID NO: 2 - protein, 11 aa, Artificial Sequence
  Description of Artificial Sequence Synthetic peptide
NCSFNITTSVR

SEQ ID NO: 3 - protein, 12 aa, Artificial Sequence
  Description of Artificial Sequence Synthetic peptide
LDIVPITNESSK

SEQ ID NO: 4 - protein, 15 aa, Artificial Sequence
  Description of Artificial Sequence Synthetic peptide
LISCNTSVLTQACPK

SEQ ID NO: 5 - protein, 32 aa, Artificial Sequence
  Description of Artificial Sequence Synthetic polypeptide
GPCINVSTVQCTHGIRPVVSTQLLLNGSLAEK

SEQ ID NO: 6 - protein, 9 aa, Artificial Sequence
  Description of Artificial Sequence Synthetic peptide
SDNFSDNAK

SEQ ID NO: 7 - protein, 6 aa, Artificial Sequence
  Description of Artificial Sequence Synthetic peptide
WNDTLK

SEQ ID NO: 8 - protein, 32 aa, Artificial Sequence
  Description of Artificial Sequence Synthetic polypeptide
TIVFNPSSGGDLEIVTHSFNCGGEFFYCNTTK

SEQ ID NO: 9 - protein, 8 aa, Artificial Sequence
  Description of Artificial Sequence Synthetic peptide
LFNSTWIR

SEQ ID NO: 10 - protein, 12 aa, Artificial Sequence
  Description of Artificial Sequence Synthetic peptide
CSSNITGLILTR

SEQ ID NO: 11 - protein, 20 aa, Artificial Sequence
  Description of Artificial Sequence Synthetic peptide
DDSNGSEILEIFRPGGGDMR

SEQ ID NO: 12 - protein, 62 aa, Artificial Sequence
  Description of Artificial Sequence Synthetic polypeptide
AYDTEVHNVWATHACVPTDPNPQELVLANVTENFNMWNNTMVEQMHEDIISLWDQSLKPCVK

SEQ ID NO: 13 - protein, 36 aa, Artificial Sequence
  Description of Artificial Sequence Synthetic polypeptide
LTPLCVTLNCTDVTNATNINATNINNSSGGVESGIK

SEQ ID NO: 14 - protein, 9 aa, Artificial Sequence
  Description of Artificial Sequence Synthetic peptide
CNNETFNGK

SEQ ID NO: 15 - protein, 11 aa, Artificial Sequence
  Description of Artificial Sequence Synthetic peptide
INCTRPNNNTR

SEQ ID NO: 16 - protein, 8 aa, Artificial Sequence
  Description of Artificial Sequence Synthetic peptide
QAHCNISR

SEQ ID NO: 17 - protein, 27 aa, Artificial Sequence
  Description of Artificial Sequence Synthetic peptide
EGNNGTWNGTIGLNDTAGNDTIILPCK

SEQ ID NO: 18 - protein, 16 aa, Artificial Sequence
  Description of Artificial Sequence Synthetic peptide
LICTTDVPWDTSWSNK

SEQ ID NO: 19 - protein, 17 aa, Artificial Sequence
  Description of Artificial Sequence Synthetic peptide
TLDDIWGSNMTWMEWER

SEQ ID NO: 20 - protein, 22 aa, Artificial Sequence
  Description of Artificial Sequence Synthetic peptide
EIDNYTSTIYTLLEEAQYQQEK

SEQ ID NO: 21 - protein, 509 aa, Artificial Sequence
  Description of Artificial Sequence Synthetic polypeptide
MRVKETQRSWPNLWKWGTLILGLVIMCNAVPVWRDADTTLFCASDAQAHVTEVHNIWATHACVPTDPNPQEIHLENVTENFNMWKNNMAEQMQEDVISLWDQSLKPCVKLTPLCVTLKCTANITITNATTRTENTTKENLIGNITDELRNCSFNVTTELRDRQRKAYALFYKLDIVPINNEANSSEYRLINCNTSVIKQACPKVSFDPIPIHYCTPAGYAILKCNDKNFNGTGPCKNVSSVQCTHGIKPVVSTQLLLNGSLAEDEIIIRSENLTDNSKNIIVHLNESVVINCTRPSNNTVKSIRIGPGQTFYRTGDIIGDIRQAYCNVNGTKWYEVLRNVTKKLKEHFNNKTIVFQQPPPGGDLEITTHHFNCRGEFFYCNTTELFNNTCVNETINNGTEGWCKGDIILPCRIKQIINLWQEVGQAMYAPPVSGQIRCISNITGIILTRDGGNGKNGTLNNETFRPGGGNMKDNWRSELYKYKVVEIEPLGIAPSRAKERVVEMKREKE

SEQ ID NO: 22 - protein, 501 aa, Artificial Sequence
  Description of Artificial Sequence Synthetic polypeptide
MRVKETQMNWPNLWKWGTLILGLVIICSAVPVWKEADTTLFCASDAKAHETEVHNVWATHACVPTDPNPQEIDLENVTENFNMWKNNMVEQMQEDVISLWDQSLKPCVKLTPPCVTLHCTNANLTKANLTNVNNRTNVSNIIGNITDEVRNCSFNMTTELRDKKQKVHALFYKLDIVPIEDNNDSSEYRLINCNTSVIKQPCPKISFDPIPIHYCTPAGYAILKCNDKNFNGTGPCKNVSSVQCTHGIKPVVSTQLLLNGSLAEEEIIIRSENLTNNAKTIIVHLNKSVVINCTRPSNNTRTSITIGPGQVFYRTGDIIGDIRKAYCEINGTEWNKALKQVTEKLKEHFNNKPIIFQPPSGGDLEITMHHFNCRGEFFYCNTTRLFNNTCIANGTIEGCNGNITLPCKIKQIINMWQGAGQAMYAPPISGTINCVSNITGILLTRDGGATNNTNNETFRPGGGNIKDNWRNELYKYKVVQIEPLGVAPTRAKRRVVEREKR

SEQ ID NO: 23 - protein, 510 aa, Artificial Sequence
  Description of Artificial Sequence Synthetic polypeptide
MRVKGIRKNYQHLWRWGIWRWGIMLLGTLMICSAVPVWKEATTTLFCASDAKAYSPEKHNIWATHACVPTDPNPQELVLGNVTEDFNMWKNNMVEQMHEDIISLWDQSLKPCVKLTPLCVTLNCTDLKNSATDTNGTSGTNNRTVEQGMETEIKNCSFNITTGIGNKMQKEYALFYKLDVVPIDSNNNSDNTSYRLISCNTSVVTQACPKTSFEPIPIHYCAPAGFAILKCNNKTFSGKGPCKNVSTVQCTHGIRPVVSTQLLLNGSLAEEEIVIRSENFTNNAKTIIVQLNESVIINCTRPNNNTRKGIHIGLGRALYATGDIIGDIRQAHCNLSSKSWNKTLQQVVRKLREQFGNKTIAFNQSSGGDQEIVKHSFNCGGEFFYCDTTQLFNSTWSSNDTWNSTGVQDNNITLPCRIKQIINMWQEVGKAMYAPPIQGLISCSSNITGLLLTRDGGTNNTNATEIFRPGGGDMRDNWRSELYKYKVVKIEPLGIAPTKAKRRVVQREKR

SEQ ID NO: 24 - protein, 490 aa, Artificial Sequence
  Description of Artificial Sequence Synthetic polypeptide
MRVRGIWKNWPQWLIWSILGFWIGNMEGSVPVWKEAKTTLFCASDAKAYEKEVHNVWATHACVPTDPNPQEMVLANVTENFNMWKNDMVEQMHEDIISLWDESLKPCVKLTPLCVTLNCTNVKGNESDTSEVMKNCSFKATTELKDKKHKVHALFYKLDVVPLNGNSSSSGEYRLINCNTSAITQACPKVSFDPIPLHYCAPAGFAILKCNNKTFNGTGPCRNVSTVQCTHGIKPVVSTQLLLNGSLAEEEIIIRSENLTNNAKTIIVHLNESVNIVCTRPNNNTRKSIRIGPGQTFYATGDIIGNIRQAHCNINESKWNNTLQKVGEELAKHFPSKTIKFEPSSGGDLEITTHSFNCRGEFFYCNTSDLFNGTYRNGTYNHTGRSSNGTITLQCKIKQIINMWQEVGRAIYAPPIEGEITCNSNITGLLLLRDGGQSNETNDTETFRPGGGDMRDNWRSELYKYKVVEIKPLGVAPTEAKRRVVEREKR

SEQ ID NO: 25 - protein, 690 aa, Artificial Sequence
  Description of Artificial Sequence Synthetic polypeptide
MRVKGIRKNCQQHLWRWGTMLLGILMICSAAENLWVTVYYGVPVWKEATTTLFCASDAKAYDTEVHNVWATHACVPTDPNPQEMVLENVTEYFNMWKNNMVEQMHEDIISLWDQSLKPCVKLTPLCVTLTCTDYEWNCTGIRNSICKYNNMTNNSSSGNYTGWERGEIKNCSFNSTISGIRDKVRKEYALLYKIDLVSIDGSNTSYRMISCNTSVITQSCPKISFEPIPLHYCTPAGFALLKCNDKKFNGTGLCHNVSTVQCTHGIKPVVSTQLLLNGSLAEEEVVIRSKNFTDNAKIIIVQLNETVEINCTRPGNNTRKSIHIAPGRTFYATGEIIGDIRRAHCNISREKWNTTLHRIATKLREQYNKTIVFNQSSGGDPEIVMHSVNCGGEFFYCNTSKLFNSTWNSTGGSISEDSENITLPCRIKQIVNMWQEVGKAMYAPPIRGQIRCSSNITGLLLTRDGGINQSISETFRPGGGDMRDNWRSELYKYKVVKIEPLGIAPTKARERVVQREKEAVGIGAVFLGFLGAAGSTMGAASLTLTVQARLLLSGIVQQQNNLLRAIEAQQHMLQLTVWGIKQLQARVLALERYLRDQQLMGIWGCSGKLICTTAVPWNASWSNKSLNDIWNNMTWMQWEREIDNYTGLIYSLLEESQNQQEKNEQDLLALDKWANLWTWFDISNWLWYIK

(19) United States
(12) Patent Application Publication
(10) Pub. No.: US 2015/0359874 A1
(43) Pub. Date: Dec. 17, 2015

(54) VACCINE FORMULATION

(71) Applicant: DUKE UNIVERSITY, Durham, NC (US)

(72) Inventors: Anthony M. Moody, Durham, NC (US); Barton F. Haynes, Durham, NC (US)

(21) Appl. No.: 14/382,711

(22) PCT Filed: Mar. 5, 2013
(86) PCT No.: PCT/US2013/029164

§ 371 Date: Sep. 3, 2014

Related U.S. Application Data
(60) Provisional application No. 61/606,881, filed on Mar. 5, 2012.

(51) Int. Cl. A61K 39/21; C12N 7/00

(52) U.S. Cl. CPC A61K 39/21; C12N 7/00; C12N 2740/16134; A61K 2039/55511

(57) ABSTRACT

The present invention relates, in general, to a method of inducing an immune response to HIV-1 in a mammal and, in particular, to a vaccine formulation suitable for use in such a method comprising an HIV-1 envelope (Env) immunogen comprising recombinant Envs with some degree of high-mannose glycan residues and a Toll-like receptor (TLR) agonist-supplemented squalene-based adjuvant.

This application claims priority from U.S. Provisional Application No. 61/606,881, filed Mar. 5, 2012, the entire content of which is incorporated herein by reference.

This invention was made with government support under Grant No. A1067854-06 awarded by the National Institutes of Health. The government has certain rights in the invention.

### TECHNICAL FIELD

The present invention relates, in general, to a method of inducing an immune response to HIV-1 in a mammal and, in particular, to a vaccine formulation suitable for use in such a method comprising an HIV-1 envelope (Env) immunogen comprising recombinant Envs with some degree of high-mannose glycan residues and a Toll-like receptor (TLR) agonist-supplemented squalene-based adjuvant.

### BACKGROUND

The primary goal of vaccination is to produce a beneficial immune response that prevents disease upon exposure to a potential pathogen. In some cases, vaccine immunogens are themselves sufficient to induce the desired response (eg, tetanus toxoid) while, in other cases, an adjuvant is required. Adjuvants are materials which, when combined with an immunogen, can enhance the immune response to that immunogen (Vaccine Design: the subunit and adjuvant approach, edited by Michael F. Powell and Mark J. Newman, Pharmaceutical Biotechnology 6: 1-28 (1995)).

Adjuvants can act through a depot effect, where an immunogen is physically retained at the site of vaccination, thereby increasing the local concentration of the immunogen that can be recognized by the immune system. In addition, adjuvants can stimulate immune defense mechanisms that recognize potential threats or damage. One example is the effect of alum adjuvant that activates the inflammasome via NLRP3 (Li et al, J Immunol. 181(1):17-21 (2008)). Furthermore, adjuvants have been shown to increase the immune response to smaller doses of an immunogen, permitting “dose sparing” when widespread vaccination programs are needed (Levie et al, J Infect Dis. 198(5):642-649 (2008)).

Toll-like receptors (TLRs) are host innate immune system cell recognition molecules to which molecules of invading pathogens can bind. Innate immune cell activation via TLRs by pathogen molecules serve to begin the activation of the adaptive immune system for production of protective T and B cell immunity. Host TLRs recognize distinct pathogen-associated molecular patterns, such as bacterial lipopolysaccharide (TLR4), as well as DNA (TLR9) or RNA (TLR7), by pattern recognition receptors (PRRs) such as TLRs (Schenten and Medzhitov, Adv. Immunol 109:87-124 (2011)). Activation of PRRs triggers cell signaling leading to activation of immediate inflammatory responses and then later adaptive T and B cell anti-pathogen responses (Schenten and Medzhitov, Adv. Immunol. 109:87-124 (2011); Olive, Expert Rev. Vaccines 11: 237-256 (2012)). Thus, addition of TLR agonists to adjuvant and/or vaccine formulations is an important strategy for enhancing vaccine induced anti-pathogen responses, and, in particular, enhancing anti-HIV protective responses.

In the past, addition of single TLR agonists (a TLR4, TLR7 or TLR9 agonist) or combinations of TLR agonists (TLR2/6, 3 and 9 agonists) to vaccines has been a strategy for enhancing vaccine efficacy (Stevceva, Curr. Med. Chem. 18: 5079-82 (2011)). Different TLR agonists induce distinct signatures of innate responses following immune stimulation (Kwissa et al, Blood 119: 2044-55 (2012)). Synergy of TLR3 and 4 agonists with TLR 7, 8 and 9 agonists has been reported for triggering of a T helper 1-type of immune response (Napolitani et al, Nature Immunology 6: 769-76 (2006)). However, to date, there have been no reports of mixtures of TLR7 plus TLR9 agonists that have either additive or synergistic effects on stimulation of antibody responses by a vaccine.

In 2009, an HIV ALVAC/AIDSVAX experimental vaccine Phase IIB efficacy trial in Thailand demonstrated an estimated 31.2% vaccine efficacy (Rerks-Ngarm et al, NEJM 361: 2209-2220 (2009)). A recent immune correlates analysis of potential protective antibody responses in the trial demonstrated an inverse correlation of HIV-1 envelope V1V2 plasma antibodies with decreased infection risk (Haynes B F, Case Control study of the RV144 trial for immune correlates: the analysis and way forward. AIDS Vaccine 2011 (Bangkok, Thailand, 2011), Haynes B F et al, N. Eng. J. Med. In press April 2012). Thus, devising adjuvant and envelope formulations that generate higher levels of Env antibodies than those seen in RV144 is a key goal of HIV vaccine development.

One type of antibody that is desirous to induce are antibodies to the HIV envelope glycans. One such antibody is the broadly neutralizing antibody 2G12 that binds primarily to high mannose residues of glycans, such as man(4), man(5), man(7) and man(8) high mannose residues (Calarese et al, PNAS USA 102:13372-7 (2005)). Thus, production of Env immunogens with high levels of expression of high mannose glycan residues for formulation with novel adjuvants is a key priority for HIV vaccine development.

Kifunensine is a plant alkaloid that inhibits glycoprotein processing. Kifunensine has been shown to promote the expression on HIV-1 Env of high-mannose glycans (Kong et al, J. Mol. Biol. 403:131-147 (2010); Scanlan et al, J. Mol. Biol. 371:16-22 (2006)).

The present invention relates, at least in part, to a formulation comprising an HIV-1 envelope protein gp120 or gp140 produced under conditions such that Env glycan expression is limited to, or essentially limited to, high mannose carbohydrate residues, and a squalene-based adjuvant comprising a mixture of, for example, a TLR7 agonist and a TLR9 agonist. The invention further relates to a method of inducing an anti-HIV-1 immune response in a mammal (e.g., a human) using same.

### SUMMARY OF THE INVENTION

In general, the present invention relates to a method of inducing an immune response to HIV-1 in a mammal. The invention further relates to a vaccine formulation suitable for use in such a method comprising an HIV-1 envelope (Env) immunogen comprising recombinant Envs with some degree of high-mannose glycan residues and a Toll-like receptor (TLR) agonist-supplemented squalene-based adjuvant.

Objects and advantages of the present invention will be clear from the description that follows.

### BRIEF DESCRIPTION OF THE DRAWINGS

FIG. 1. Adjuvant panel (8 variations).

FIG. 2. 63521 gp140 peak II purified.

FIG. 3. BN-PAGE of 63521 gp140 gD—fractions from HPLC.

FIG. 4. Antigenicity of 63521 Env protein.

FIG. 5. Midpoint binding Titers of Rhesus macaque plasma from Env 63521.B+STS adjuvant-immunized animals to 63521.B transmitted/founder virus Env.

FIG. 6. Induction of rhesus monkey plasma mAb A32 blocking antibodies by B.63521 Env in various adjuvant formulations. A32 ab binds to the CI region of gp120 and is a potent mediator of antibody dependent cellular cytotoxicity (ADCC).

FIG. 7. sCD4 blocking×JRFL.

FIG. 8. 50% Neutralization titers against 92BR025.B.

FIG. 9. 50% Neutralization titers against SF162.B.

FIG. 10. Monkey study #32 B.63521 gp140C immunization. Post immunization #4 or 5.

FIG. 11. 63521 gp140C gDneg 293 KIF “Peak 2” 110831.

FIG. 12. 63521TC1 gp140: without Kifunensine.

FIG. 13. 63521_TC21 gp140: with Kifunensine.

FIG. 14. 63521.B-KIF envelope binds mAbs A32, sCD4 and T8 in response to A32 and sCD4 triggering upregulates the CCR5 co-receptor binding site (17b) and also expresses the glycan high mannose broad neutralizing antibody (BnAb) binding site defined by mAb 2G12.

FIG. 15. CD4 binding site BnAb 1b12 binding site is also available on 63521.B-KIF Env.

FIG. 16. V2V3 quaternary BnAb binding site is on both the A244Delta 11 gp120 and on the non-KIF treated 63521.B Env but not on KIF treated 63521.

FIG. 17. V1V2 mabs 697d and 2158 bind to all three Envs A244 Delta 11 gp120, and to KIF treated and non-treated 63521.E gp140. CD4 BS antibody VRC01 also binds to all three Envs.

FIG. 18. Kifunensine (KIF) treatment does upregulates the binding of 2G12 to 63521.B gp140C, while the binding of the CD4 binding site antibody 1b12 is minimally altered.

FIG. 19. Sequences.

FIGS. 20A-20E. Oil-in-water emulsion adjuvants combined with Env immunogens elicit HIV-1 Env-reactive and V1V2-directed antibodies. (FIG. 20A) All animals developed antibodies against gp140 B.63521; after 5 immunizations, STS elicited the lowest endpoint titer (1:1,905; 95% CI 1:728-1:4,989), STS+oCpG+R848 elicited the highest endpoint titer (1:25,704; 95% CI 1:5,420-1:121,899; t-test p=0.004). (FIG. 20B) Binding to case A2 V1V2-gp70; STS elicited the lowest endpoint titer (1:19,890; 95% CI 1:912-1:434,011), STS+oCpG+R848 elicited the highest titer (1:298,498; 95% CI 1:44,722-1:1,992,000). Similar binding patters were observed against V1V2 tags representing clades A, CRF01_AE, and C (FIGS. 20C, 20D, and 20E, respectively).

FIGS. 21A-21D. Plasma antibodies block the binding of mAbs and sCD4. Plasma antibodies blocked binding of labeled ligands to Env proteins. Binding of sCD4 (FIG. 21A) and mAb b12 (FIG. 21B) to gp140 B.JRFL was inhibited by immune plasma; titers were lowest for STS and highest for STS+oCpG+R848. Blocking of ADCC-mediating mAb A32 was lowest for STS and highest for STS+R848 (FIG. 2I C). Low level blocking of broadly neutralizing mAb CHOI was found in the STS+oCpG+R848 immunized group (FIG. 21D).

FIGS. 22A and 22B. Plasma neutralization. Neutralization titers with 50% activity against B.BaL (FIG. 22A) and B.BX08 (FIG. 22B). After four immunizations, the titer elicited against B.BaL by STS was 1:45 vs. STS+oCpG+R848 at 1:374 (t-test p<0.05). Titers against B.BX08 were 1:59 and 1:216, respectively at the same time points (t-test p<0.05).

FIGS. 23A and 23B. Plasma ADCC activity. (FIG. 23A). ADCC plasma titer against B.BaL coated target cells after five immunizations was lowest for STS (1:2,317, 95% CI 1:579-1:9,268) and highest for STS+oCpG+R848 (1:47,753, 95% CI 1:27,227-1:83,946; t-test p=0.001). (FIG. 23B) ADCC peak activity was lowest for STS at 14.9%±0.9% and highest for STS+oCpG+R848 at 31.5%±0.9% (t-test p=0.0002).

FIGS. 24A-24D. Cytokine/chemokine stimulation by TLR agonists in oil-in-water emulsion. (FIG. 24 A) CXCL10 (IP-10) was elevated in 3/3 animals immunized with STS+oCpG+R848, peaking at 24 hours and returning to baseline by one week. One of 3 animals immunized with STS+oCpG had an elevation at baseline, peaked at 24 hours. and waned at later points. (FIG. 24B) IFN-γ was transiently elevated in 2/3 animals immunized with STS+oCpG+R848, peaking at 24 hours. (FIG. 24C) IL-6 was elevated in 2/3 animals immunized with STS+oCpG and in 1/3 animals immunized with STS alone; the peak occurred at 6 hours and returned to baseline by 24 hours. (FIG. 24D) IL-12 showed a non-specific pattern over the course of the study. This lack of a pattern was observed for 15 other chemokines/cytokines (data not shown).

### DETAILED DESCRIPTION OF THE INVENTION

The present invention relates to a method of inducing an immune response to HIV-1 in a mammal (e.g., a human). The invention further relates to a vaccine formulation suitable for use in such a method comprising an HIV-1 envelope (Env) immunogen that includes recombinant Envs with some degree of high-mannose glycan residues (preferably greater than 90%), and Toll-like receptor (TLR) agonist-supplemented squalene-based adjuvant.

The recombinant Envs suitable for use in the invention can be produced, for example, in the presence of an agent (such as kifunensine or swansonine) that inhibits production of complex glycans and promotes expression on the surface of the Env of high mannose glycans to which HIV-1 neutralizing antibodies can bind. Suitable Envs can also be produced in cell types that result in expression on the surface of the Env of high mannose glycans. (See, for example, Kong et al, J. Mol. Biol. 403:131-147 (2010); Scanlan et al, J. Mol. Boil 371: 16-22 (2006).) Transmitted/founder Envs are preferred.

Transmitted/founder HIV-1 strains have been described that represent the precise viral species that traversed mucosal barriers to establish HIV-1 infection (Keefe et al, PNAS (USA) 105:7552-57 (2008)). Transmitted/founder envelopes have also been described as immunogens (WO 2011/106100). Described below is the use of the 63521 clade B transmitted/founder Env oligomer formulated with a TLR agonist-supplemented squalene based adjuvant for the induction of anti-Env binding and neutralizing antibodies.

The present invention relates, in part, to an adjuvant that has a base composition similar to MF-59 but differs, for example, by use of phosphate buffered saline instead of distilled water (Ott et al, Vaccine 13(16):1557-1562 (1995), Vogel and Powell, in Vaccine Design: the subunit and adjuvant approach, edited by Michael F. Powell and Mark J. Newman, Pharmaceutical Biotechnology 6: 141-228, (1995)) (see also U.S. Pat. No. 5,709,879 and U.S. Pat. No. 6,451,352). The adjuvant can be combined with TLR agonists (e.g., TLR 7, TLR7/8 and TLR 9 agonists) that trigger specific immune responses (Kwissa et al, Blood 119:2044-55 (2012), Horscroft et al, J. of Antimicrobial Chemotherapy epub ahead of print, Jan. 18, 2012 doi: 10:1093/jac/dkr588)).

Specifically, the adjuvant can comprise an oil-in-water emulsion based on isotonic phosphate buffered saline that is combined with specific agonists for TLRs that are present on mammalian immune cells. The preferred properties of the adjuvant mixture are as follows.

1. The base adjuvant composition comprises:

a. Phosphate buffered saline, pH 7.4 (1.06 mM monobasic potassium phosphate [KH₂PO₄], 2.97 mM dibasic sodium phosphate [Na₂HPO₄], 155 mM sodium chloride [NaCl], in aqueous solution)—selected as an isotonic base that would be less irritating to tissues when injected or applied topically;

b. Squalene—a naturally occurring oil that is a biological precursor of cholesterol and that is found in all animal species;

c. Polysorbate 80 (Tween 80)—a nonionic emulsifier;

d. Sorbitan trioleate (SPAN 85)—a nonionic emulsifier.

2. The added TLR ligands consist of one or more of:

a: Purified, detoxified lipid A derived from Salmonella Minnesota R595 (a TLR-4 ligand) (this is from Sigma Chemicals);

b. 1-[4-amino-2-(ethoxymethyl)imidazo[4,5-c]quinolin-1-yl]-2-methylpropan-2-ol (resiquimod, R848; a TLR-7/8 ligand) (Pockros et al, Gastroenterology 124:A766 (2003), Pockros et al, J. Hepatol. 47(2):174-182 (2007)).

c. Oligonucleotide 5′-TGCTGCTTTTGTGCTTTTGTGCTT-3′ (ODN 10103, type B oCpG; a TLR-9 ligand). (Vacari et al, Antiviral Therapy 12:741-751 (2007)-ACTILON).

The base adjuvant composition (STS) can be prepared by combining 5% (volume-to-volume) squalene, 0.5% (v/v) polysorbate 80, and 0.5% (v/v) sorbitan trioleate in isotonic phosphate buffered saline. This material can be mixed, for example, using a benchtop homogenizer for 5 minutes at room temperature, followed by emulsification using a Microfluidizer M-110S with the circulation coil immersed in an ice water bath. The Microfluidizer can be primed three times with the same adjuvant mixture that is to be homogenized in order to equilibrate the system; each priming pass can use sufficient volume (8 mL) to completely fill the chamber and coil. Each batch of adjuvant can be passed through the emulsifier five times at 15000 psi prior to collection. Final adjuvant batches can be kept at room temperature prior to mixing with the immunogen.

Formulations of adjuvant mixtures containing the TLR ligands (2a-c above) can be prepared in the exact same fashion, using the same priming and production procedures. The final concentrations of TLR ligands used can be as follows:

2. Final concentrations of added TLR ligands.

a. 0.2 mg/mL of purified, detoxified lipid A derived from Salmonella Minnesota R595;

b. 1 mg/mL of 1-[4-amino-2-(ethoxymethyl)imidazo[4,5-c]quinolin-1-yl]-2-methylpropan-2-ol;
c. 6.67 mg/mL of oligonucleotide 5′-TGCTGCTTTTGTGCTTTTGTGCTT-3′.

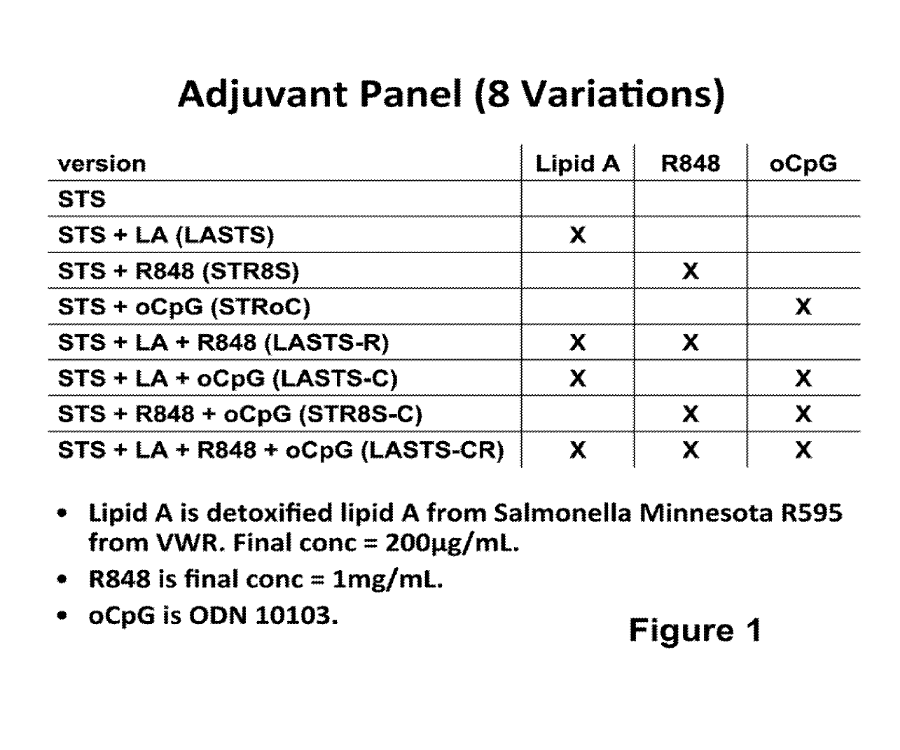

For each preparation where multiple TLR ligands are used, the final concentration of each component can be as indicated above (see FIG. 1).

The mode of administration of the formulation described herein can vary, for example, with the specific immunogen, the patient (human or non-human mammal) and the effect sought, similarly, the dose administered. Generally, administration will be subcutaneous or intramuscular. Optimum dosage regimens can be readily determined by one skilled in the art.

Certain aspects of the invention are described in greater detail in the non-limiting Examples that follows. (See also PCT/US2012/000570, U.S. Pat. No. 7,485,452, U.S. Pat. No. 7,993,659, U.S. Pat. No. 7,611,704, U.S. application Ser. No. 11/812,992, filed Jun. 22, 2007, U.S. application Ser. No. 11/785,077, filed Apr. 13, 2007, PCT/US2006/013684, filed Apr. 12, 2006, PCT/US04/30397 (WO2005/028625), WO 2006/110831, WO 2008/127651, WO 2008/118470, U.S. Pub. Applns 2008/0031890 and 2008/0057075, U.S. application Ser. No. 11/918,219, filed Dec. 22, 2008, U.S. Prov. Appln No. 61/282,526, filed Feb. 25, 2010, U.S. Prov. Appln No. 61/322,725, filed Apr. 9, 2010, U.S. Prov. Appln No. 60/960,413, filed Feb. 28, 2007, and U.S. Prov Appln Nos. 61/166,625, 61/166,648 and 61/202,778, all filed Apr. 3, 2009, the entire contents of which are incorporated herein by reference. Additionally, see WO 2011/106100 and http://www.hiv.lan1.gov/content/sequencc/HIV/mainpage.html, the entire contents of which are also incorporated herein by reference.)

### Example 1

### Non-Human Primate Testing of 63521.B gp140 With Adjuvant Formulations

### Experimental Details

Testing of adjuvant combinations in non-human primates.
Animals: Twenty one adult rhesus monkeys (Macaca mulatta) were used in this study. All animals were housed at BioQual (Rockville, Md.) and maintained in accordance with the Association for Accreditation of Laboratory Animal Care guidelines at the National Institutes of Health.
Isolation of plasma and peripheral blood mononuclear cells (PBMC): EDTA anti-coagulated blood from immunized monkeys was centrifuged over Ficoll (Ficoll-Paque) and plasma and PBMC layers were collected in separate tubes. PBMC were washed in IX PBS containing 2% FBS.
Testing of antibody binding: Antibody binding assays were performed as described (Liao et al, JEM 208: 2237-49 (2011)). Antibody blocking assays were performed as described (Alam et al, J. Virol. 82: 115-25 (2007)).

### Results

FIG. 1 shows adjuvants that can be made according to the formulation strategies herein.

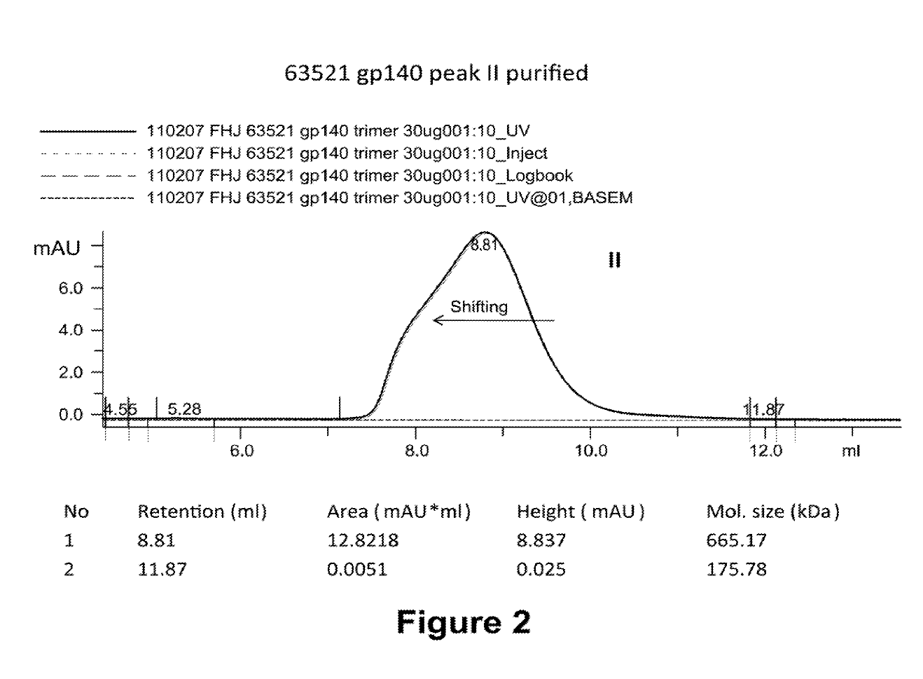

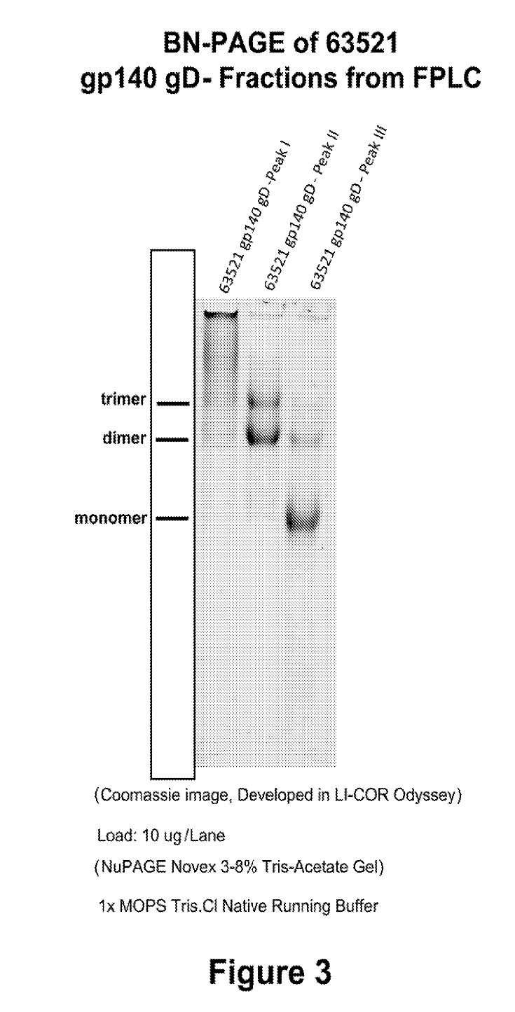

FIG. 2 shows the shifting peak of 63521.B dimers and trimers when purified on HPLC. The dimers and trimers are in equilibrium with each other. FIG. 3 shows blue native (BN) PAGE of peak II of 63521.B from HPLC.

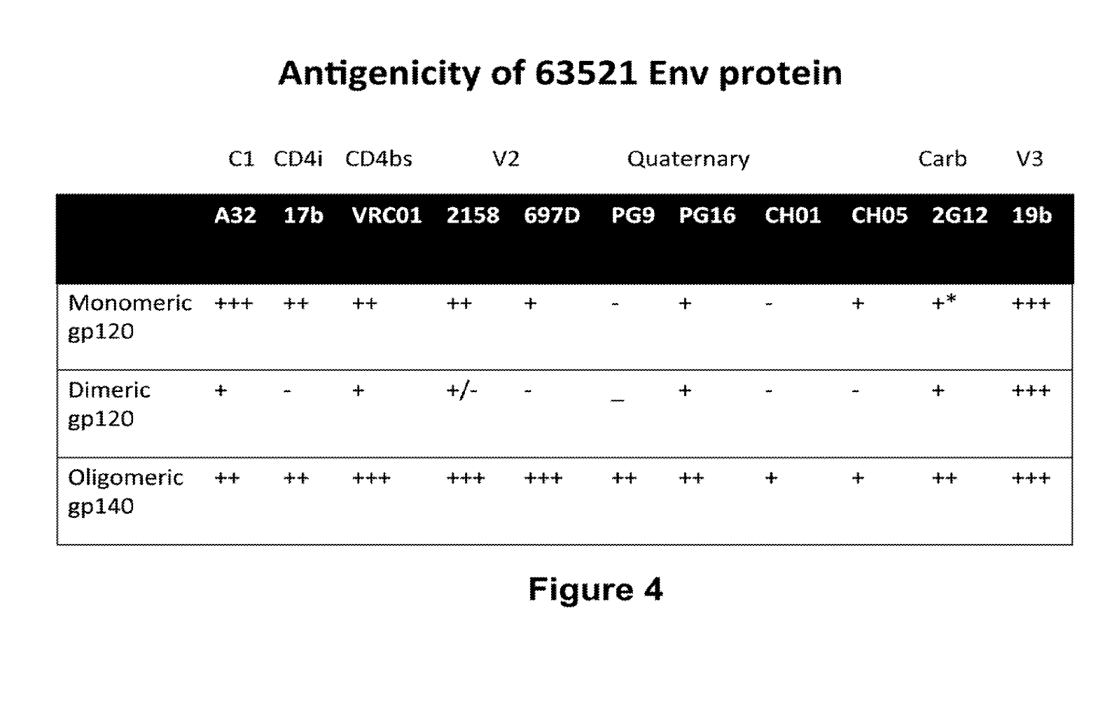

FIG. 4 shows summary of the antigenicity of 63521.B Envs as determined by surface plasmon resonance. Methods used are as described by Liao et al (JEM 208: 2237-49 (2011)).

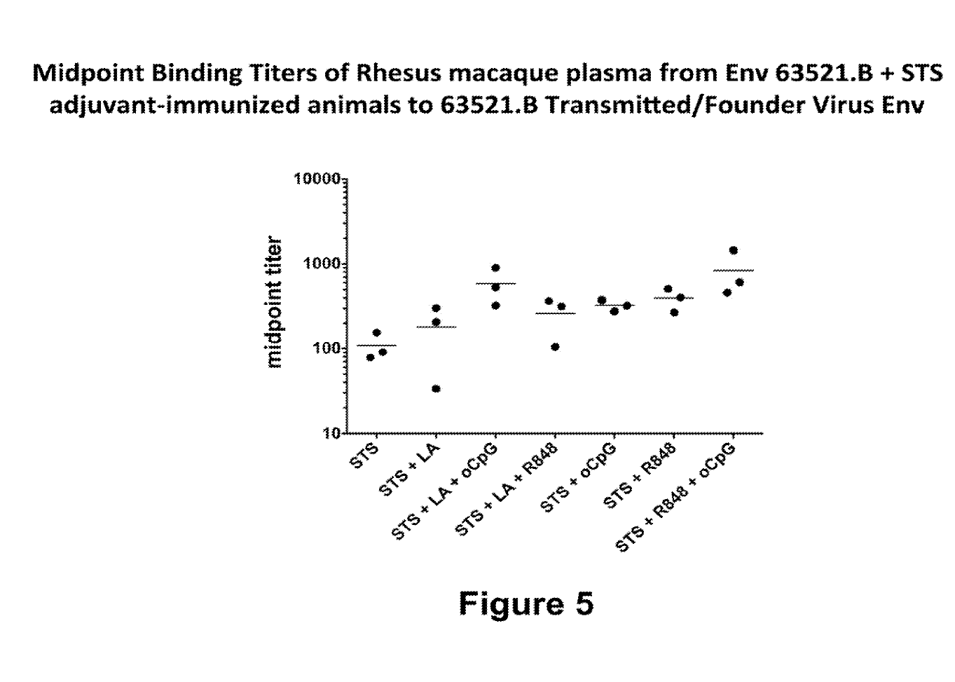

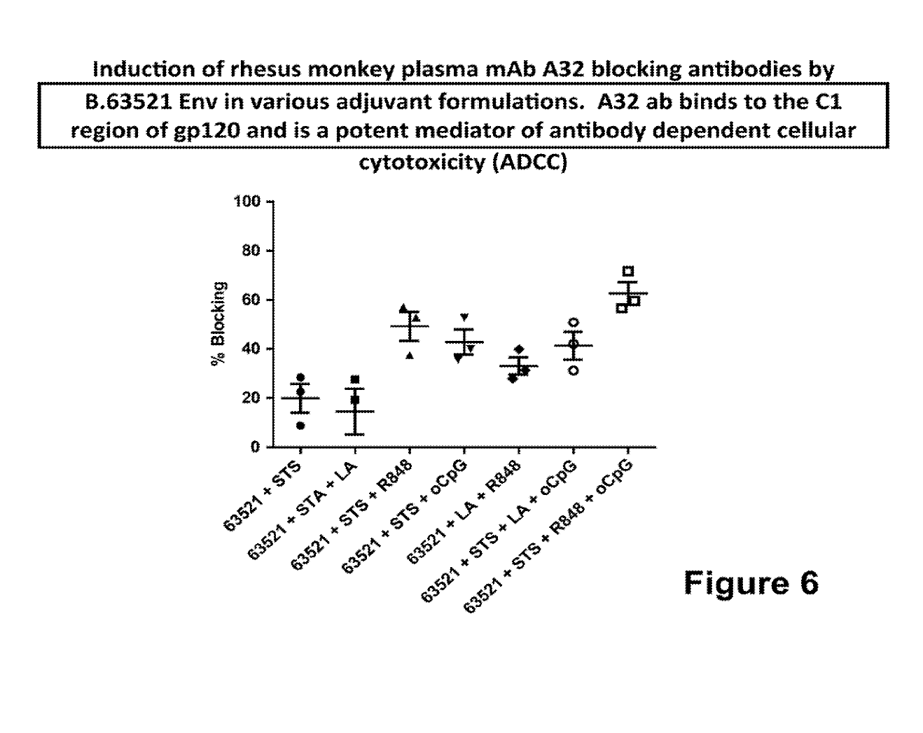

FIG. 5 shows midpoint ELISA binding titers of rhesus macaque plasma from 63521.B gp140C Env immunized animals with the Env formulated with the adjuvants listed in the graph. STS+R848+oCpGs (STR8S-C) and STS+LA+oCpGs (LASTS-C) were optimal. FIG. 6 shows that STS+R848+oCpGs (STR8S-C) was optimal for inducting blocking antibodies for the ADCC-mediating mAb A32 (Ferrari et al, J. Virol. 85:7029-36 (2011)).

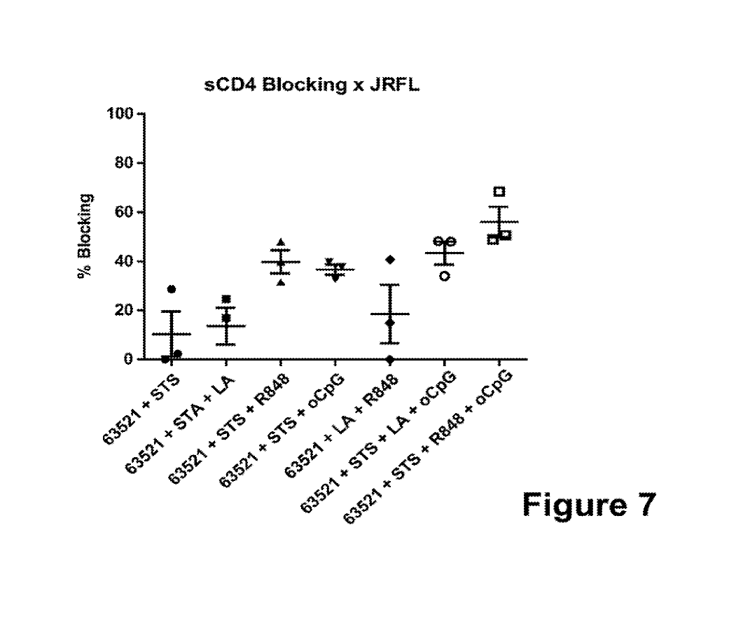

FIG. 7 shows similarly STS+R848+oCpGs (STR8S-C) was optimal for formulation with 63521.B gp140C HIV Env for induction of antibodies capable of blocking the binding of soluble (s) CD4 to HIV Env JRFL.B gp140.

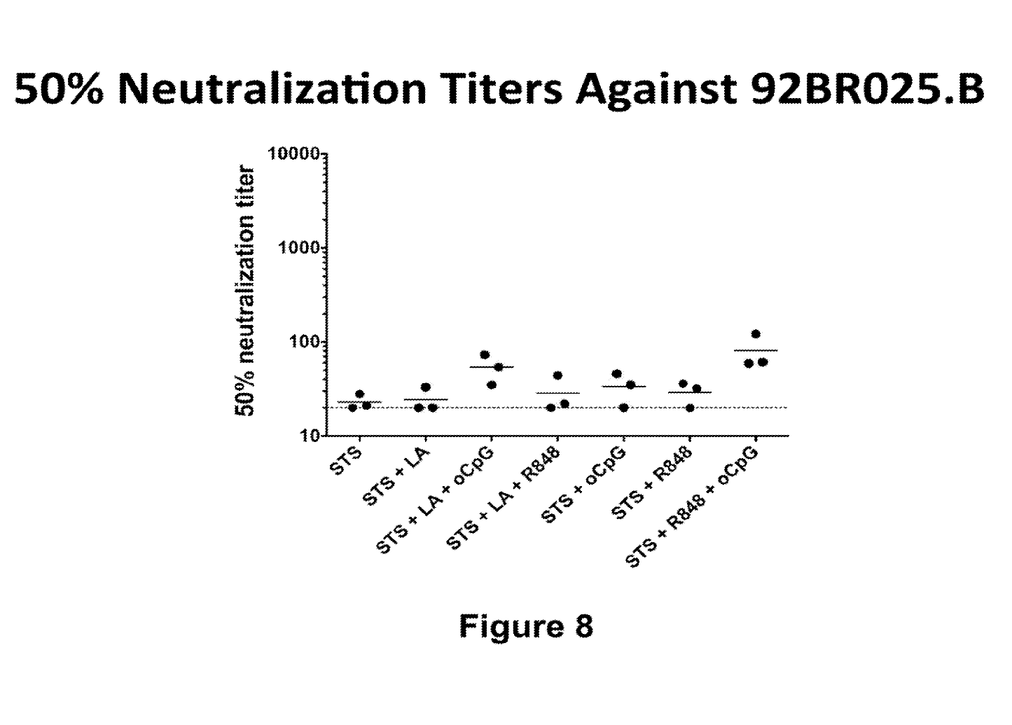

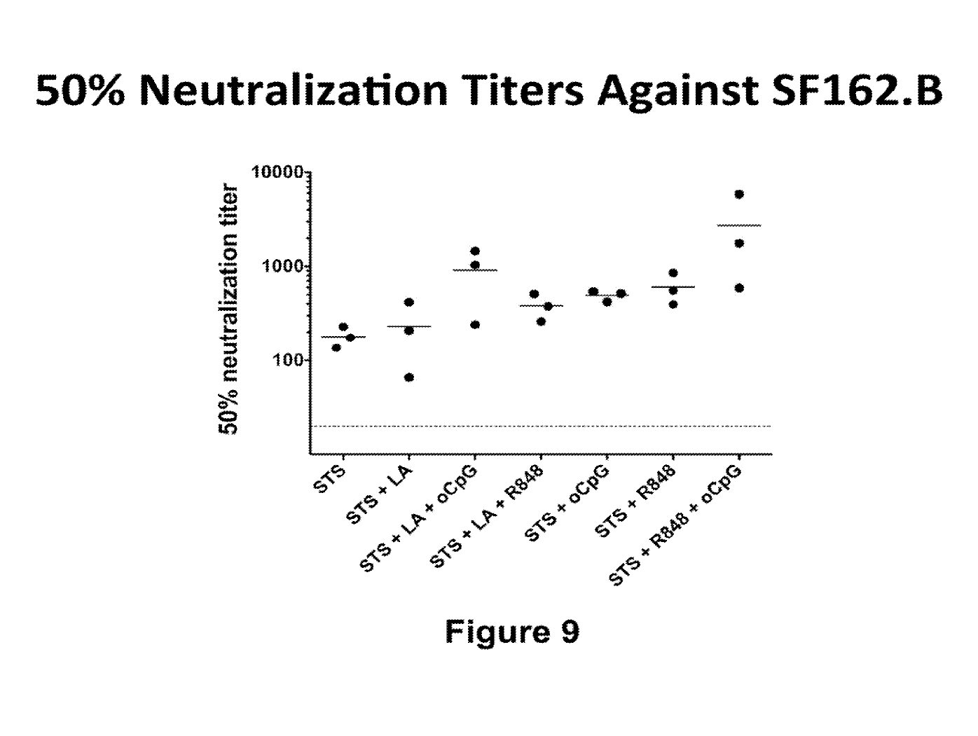

FIG. 8 shows STS+R848+oCpGs (STR8S-C) and STS+LA+oCpGs (LASTS-C) were optimal for induction of HIV neutralizing antibodies against HIV strain 92BR025.8 after three immunizations. FIG. 9 shows STS+R848+oCpGs (STR8S-C) and STS+LA+oCpGs (LASTS-C) were optimal for induction of HIV neutralizing antibodies against HIV strain SF162.B after three immunizations.

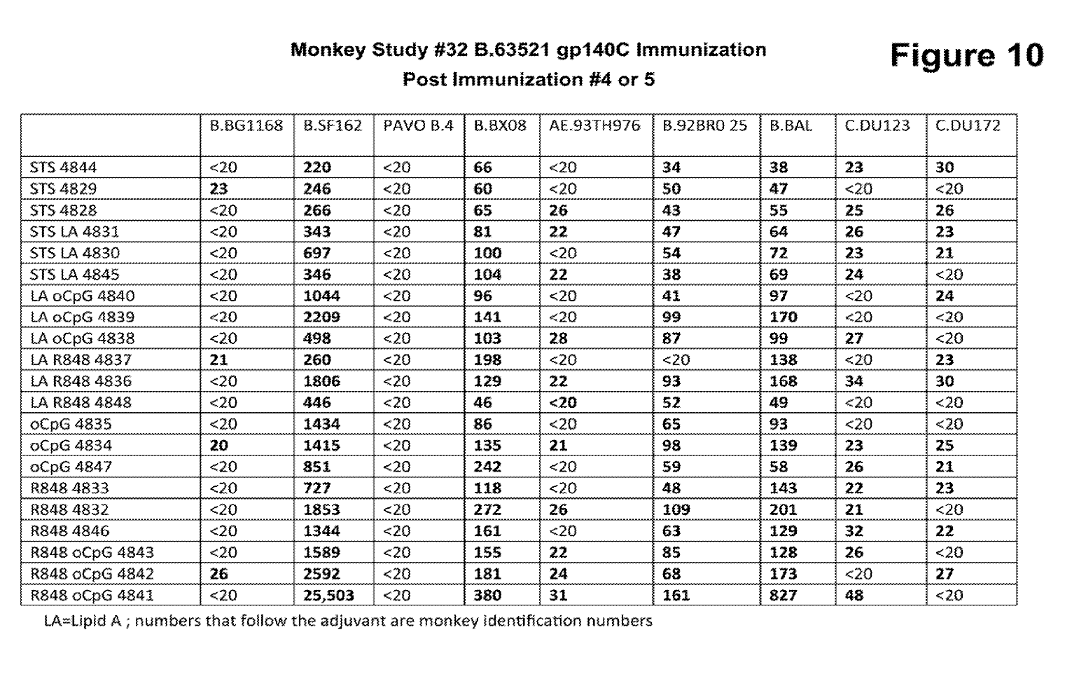

FIG. 10 provides a summary of neutralization data after either the 4^{th} or 5^{th} immunization with 63521.B gp140C env. In general, STS+R848+oCpGs (STR8S-C) and STS+LA+oCpGs (LASTS-C) were optimal,

As can be seen in FIGS. 5-10, the best adjuvant formulation of 63521.B gp140C oligomer was STR8S-C which contains the TLR-7 agonist R848 and the TLR-9 agonist the 10103 oligonucleotide CpG. Thus, STR8S-C in FIG. 1 can be formulated with the Envs in FIG. 1 or, alternatively, with the gp120 or gp140 Envs listed below with the following characteristics.

Currently, a frequent criterion for Env selection for human vaccine trials is based solely on availability and on ease of production as a GMP-produced recombinant protein. Thus, a critical need for the HIV-1 vaccine development field is provision of a number of candidate Env immunogens, chosen by rational criteria, for evaluation in Phase I human clinical trials in order to have useful human immunogenicity data for down-selection of Env boosts for vector priming immunizations in the next generation of human Phase III efficacy trials.

Over the past 4 years, CHAVI has expressed approximately 30 chronic, consensus or transmitted/founder Envs, and established criteria for envelope down-selection for consideration for use in future human clinical trials (Haynes B F, Case Control study of the RV144 trial for immune correlates: the analysis and way forward. AIDS Vaccine 2011 (Bangkok, Thailand, 2011), Haynes B F et al, N. Engl. J. Med. In press April 2012). CHAVI Env down-selection criteria are: a) antigenicity, b) binding to reverted unmutated ancestors of the types of antibodies a vaccine is desired to induce, c) immunogenicity in small animals or non-human primates, and d) ease of expression. From this work have come the selection of 5 HIV-1 envelopes with superior antigenicity, immunogenicity, reactivity with clonal lineage intermediates, and ease of expression as recombinant envelopes for GMP production. Thus, for the first time, a rational down-selection process has been carried out for Env selection for human clinical trials. (See Table 1 below.)

| | BnAb | | | | |
|---|---|---|---|---|---|
| | Anti- | RUA/IA | RUA/IA | Immuno- | Expres- |
| Envelope^{a} | gencity | V1V2^{b} | CD4BS^{c} | gencity^{d} | sion |
| gp120 | | | | | |
| B.6240Δ11 | 4+ | 3+ | 3+ | 3+ | Monomer |
| C.1086Δ7 | 3+ | 2+ | 3+ | 3+ | Monomer |
| E.427299Δ11 | 4+ | 3+ | 3+ | NA | Monomer |
| E.A244 Δ□□ | 4+ | 4+ | 3+ | 2+ | Monomer |
| gp140C | | | | | |
| B.9021 | 4+ | 4+ | 3+ | 3+ | Trimer |

CHAVI Criteria for Env Selection
^{a}Transmitted/Founder Envs B.6240, C.1086, E.427299, B.9021
^{b}V1V2 Reverted Unmutated Ancestors (RUAs) and Intermediate Antibodies (IAs); Studied: 697D, CH58, CH59, PG9, PG16, CH01-CH04
^{c}CD4BS RUAs/IAs Studied: CH30-CH34
^{d}Immunogencity in NHPs or small animals (guinea pigs).
BnAb Antigencity
4+ expressed V1V2 BnAB, CD4BS Bnab, N332 glycan BnAb Epitopes.
3+ = Expressed N332 glycan BnAbs and CD4 BS BnAbs Epitopes.
RUA/IA V1V2 Reactivity
4+ = RUAs IAs of 697D and Bnabs Ch01-CH04 are reactive.
3+ = RUAs or IAs of 697D, CH01-CH04, CH58, CH59, but RUAs non-reactive with CH01-CH04.
2+ = CH58, CH59, RUAs reactive
CH30-31 CD4BS clonal lineage RUA/IA reactivity
3+ = IAs all reactive, RUAs non-reactive
Immunogenicity
NA = Not Available
3+ = Tier 1 and weak Tier 2 Nabs induced
2+ = Tier 1 and induced

Thus, Env immunogens that can be used as monovalent primes or boosts include:

B.6240Δ11 gp120

C.1086Δ7 gp120

E.427299Δ11 gp120

E.A244 gp120Δ11

B.9021 gp140C

Alternatively, STR8S-C can be formulated with the following envs in a polyvalent mixture:

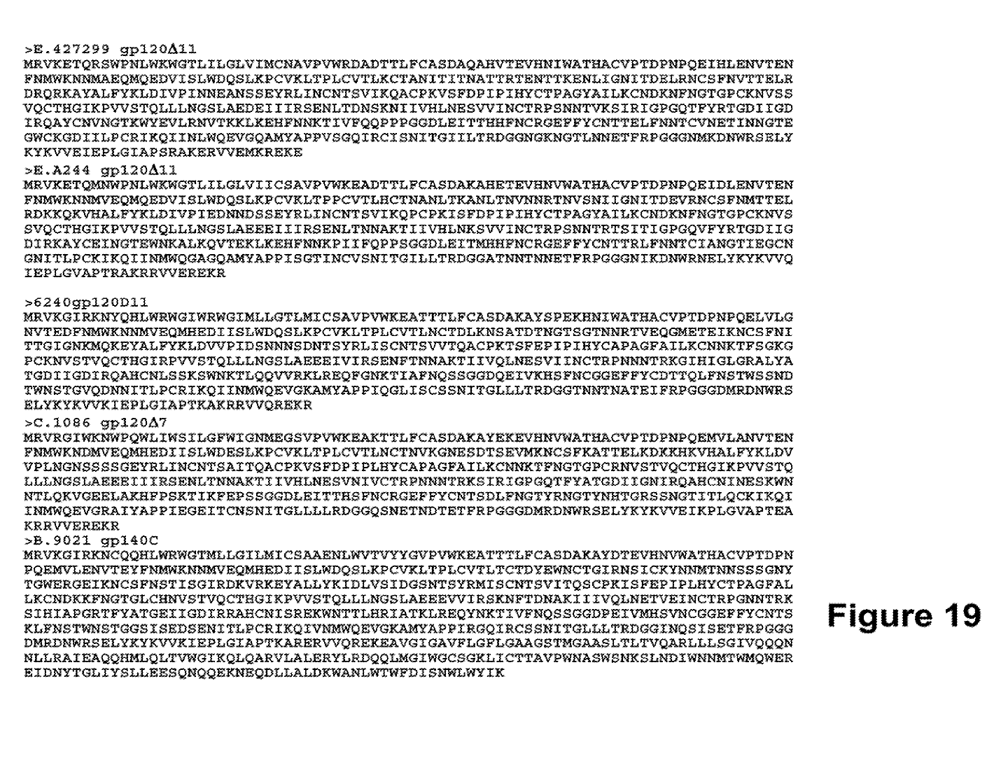

B.6240Δ11 gp120 (20 μg); C.1086Δ7 gp120 (20 μg); E427299Δ11 gp120 (20 μg); E.A244 gp120Δ11 (20 μg); and B.9021 gp140C (20 μg) together in a polyvalent mixture. (See sequences in FIG. 19.)

Alternatively, other envelopes can be used with the adjuvant STR8S-C that are found and selected based on the criteria above.

### Example 2

### Production of a Kifunensine-Treated Transmitted Founder Env Immunogen 63521.B Gp140-KIF with Selective Expression of High Mannose Glycans

The goal of this study was to make a kifunensine treated transmitted/founder recombinant envelope for use as an immunogen with the preferred adjuvant (STR8S-C in FIG. 1). Transmitted/founder envelope 63521.B (Keele et al, PNAS USA 105:7552-7557 (2008)) was expressed in 293F cells in the presence of 50 μM of kifunensine as described (Scanlan et al, J. Mol. Biol. 371:16-22 (2006), Kong et al, J. Mol. Biol. 403:131-147 (2010)).

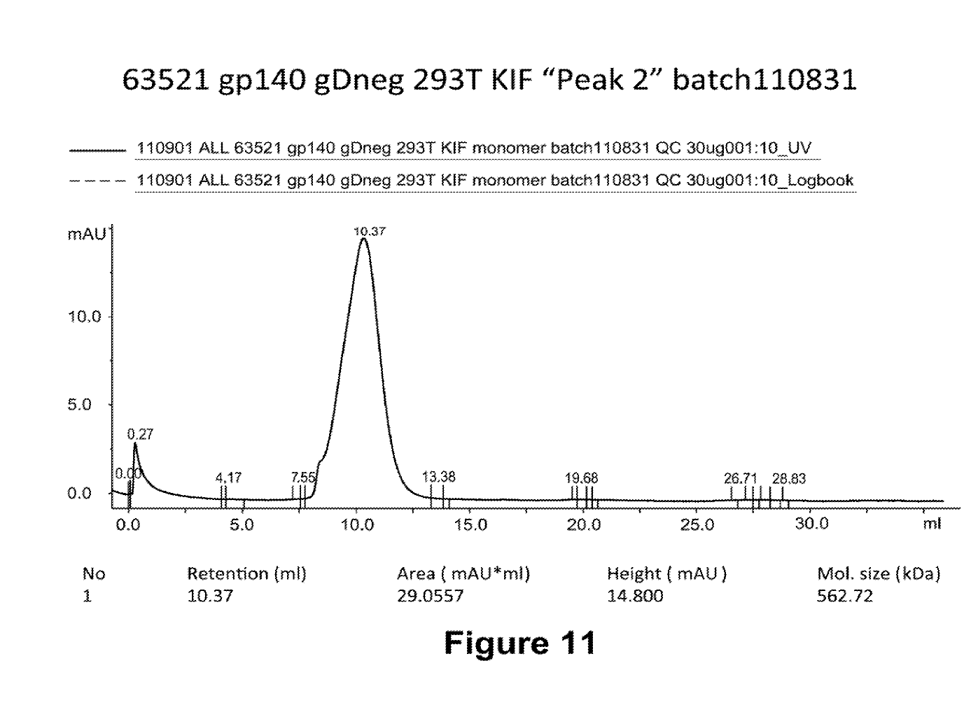

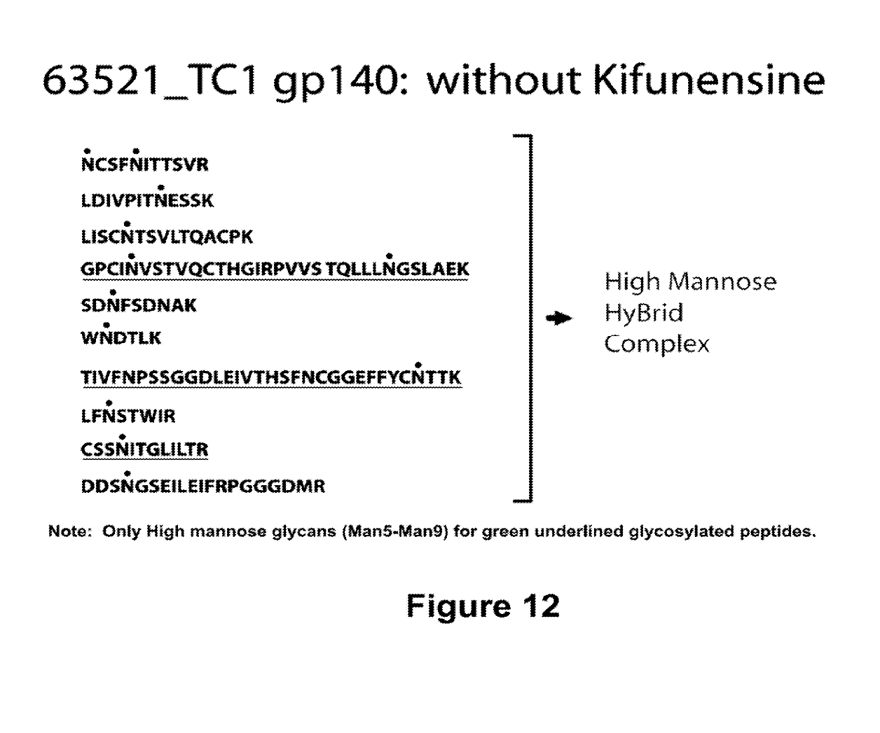

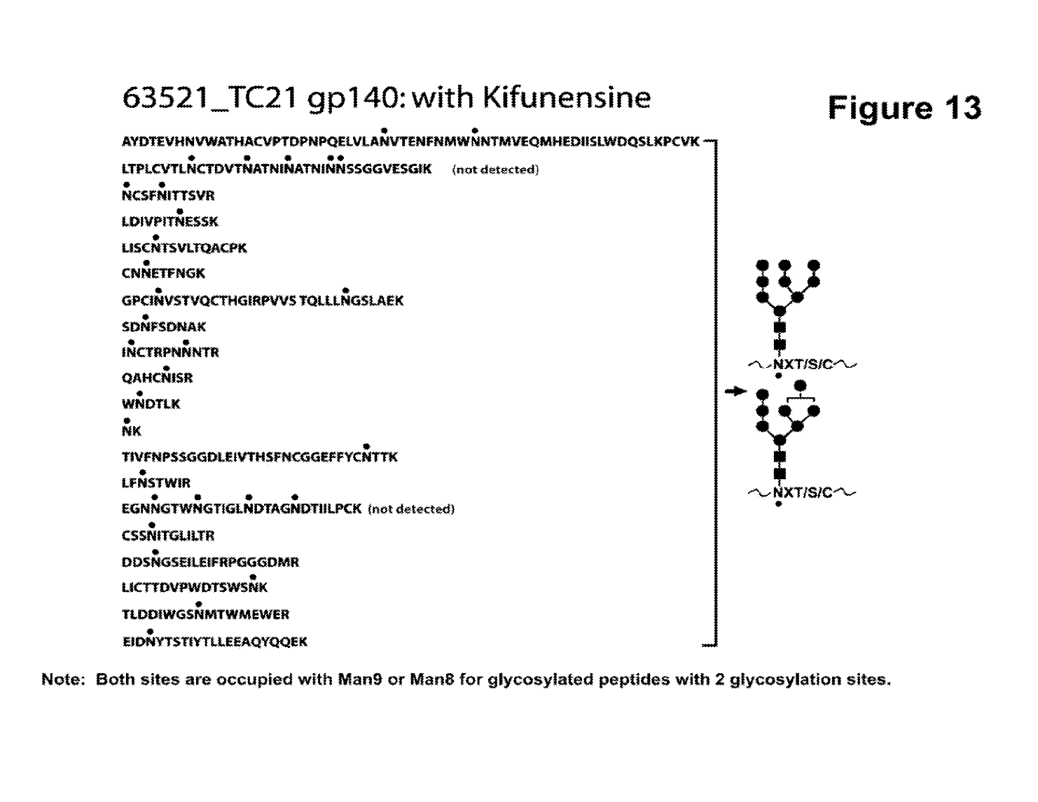

FIG. 11 shows the purification of Env 63521.B grown in kifunensine (63521.B-KIF) using HPLC. FIG. 12 shows the summary of the locations of complex vs. high mannose glycans of 63521.B Env expressed in 293F cells in the absence of kifunensine, and FIG. 13 shows that 63521.B expressed in 293F cells in the presence of kifunensine are primarily high mannose glycans. In both figures, red (dotted) N (asparagine) amino acids denote N-linked glycan sites. Methods for determining the site-specific glycans were performed as described (Go et al, J. Virology 85:8270-84 (2011)).

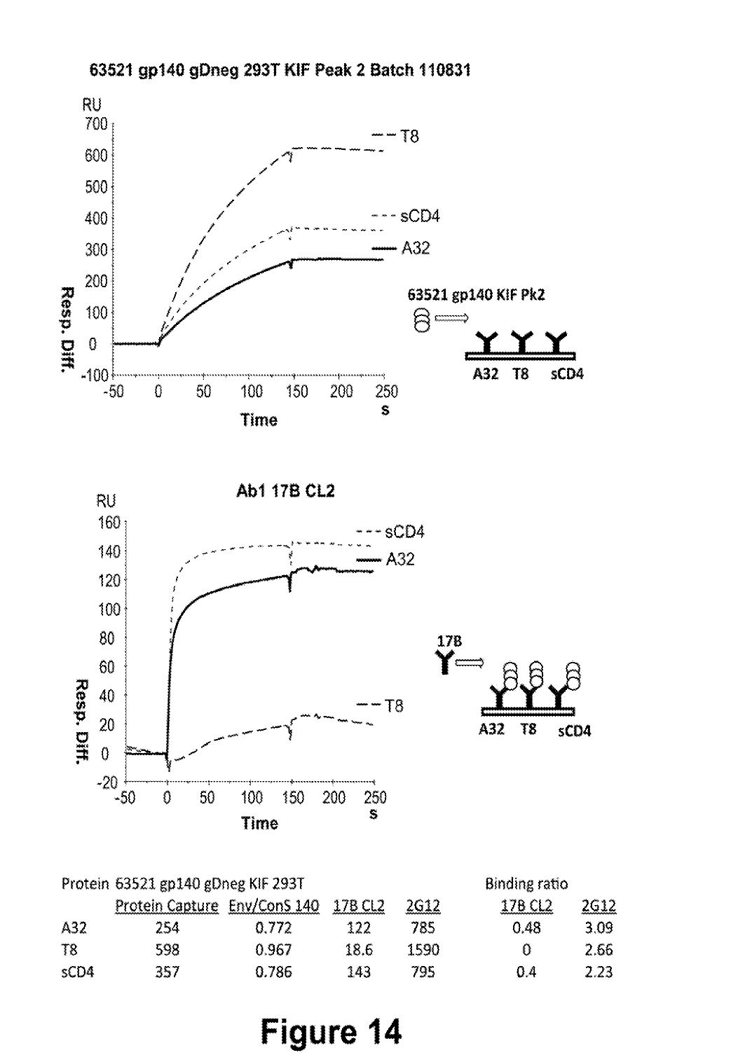

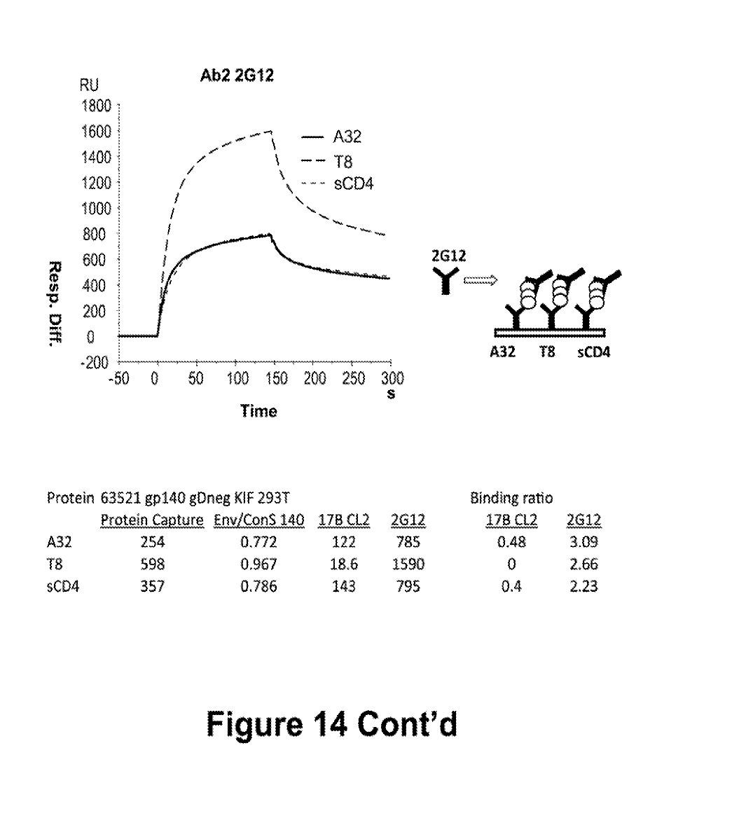

FIG. 14 shows that 63521.B-KIF envelope binds mAbs A32, sCD4 and T8, in response to A32 and sCD4 triggering upregulates the CCR5 co-receptor binding site (17b) and also expresses the glycan high mannose broad neutralizing antibody (BnAb) binding site defined by mAb 2G12. This exposure of the 2G12 glycan binding site is upregulated by sCD4 Env binding.

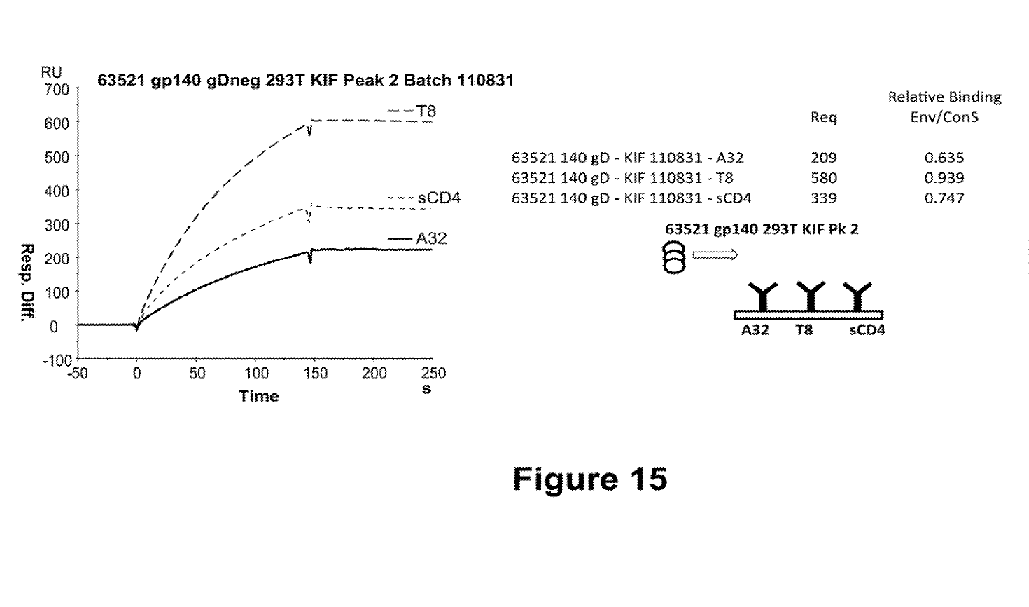

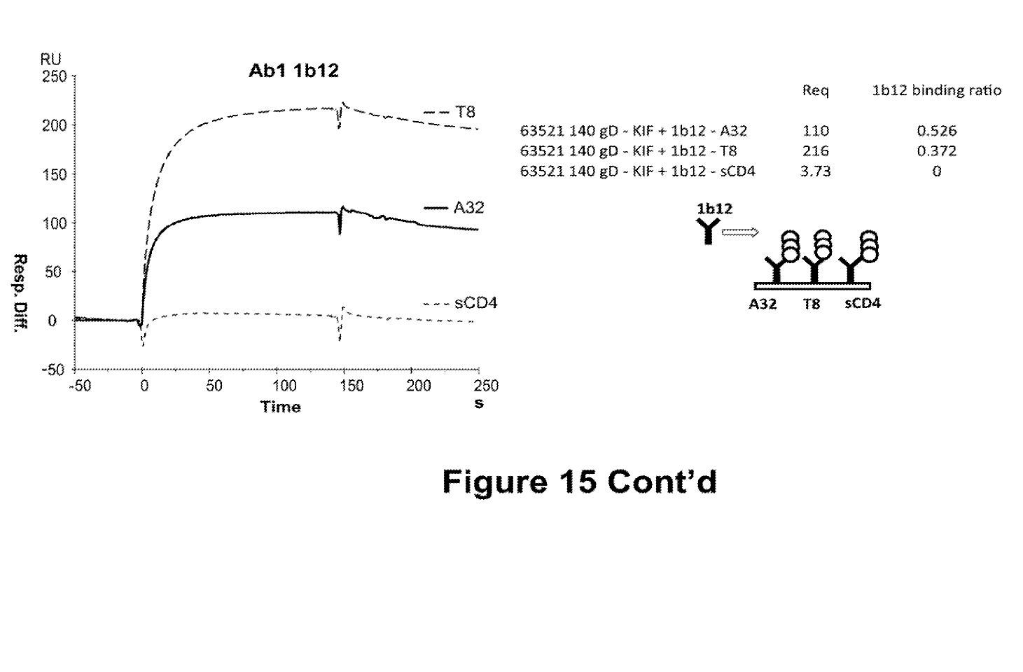

FIG. 15 shows that the CD4 binding site BnAb 1b12 binding site is also available on 63521.B-KIF Env.

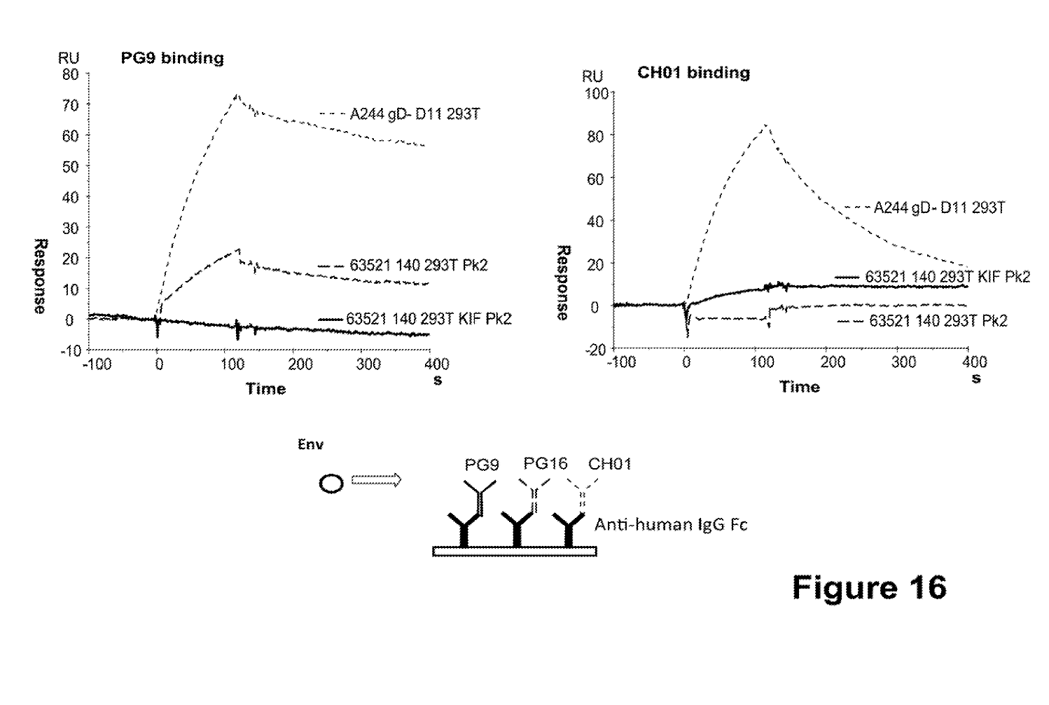

FIG. 16 shows that the V2V3 quaternary BnAb binding site is on both the A244Delta 11 gp120 and on the non-KIF treated 63521.B Env but not on KIF treated 63521. The CHOI V2V3 BnAb binds only to the A244Delta 11 gp120 Env and not to either version of 63521.B Env.

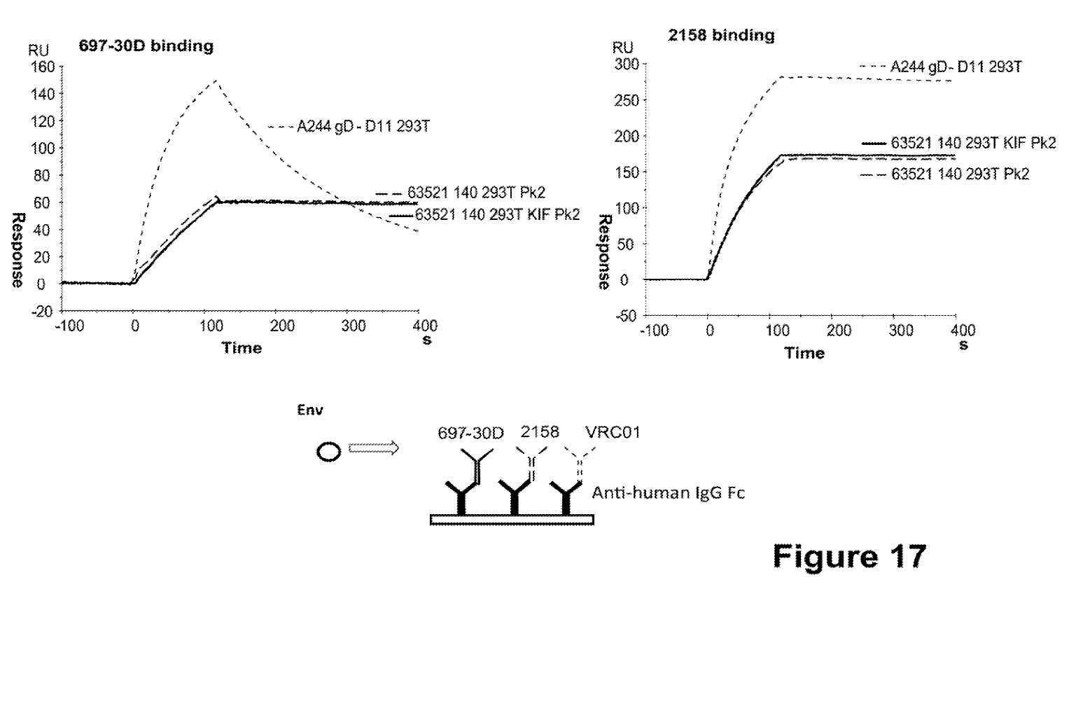

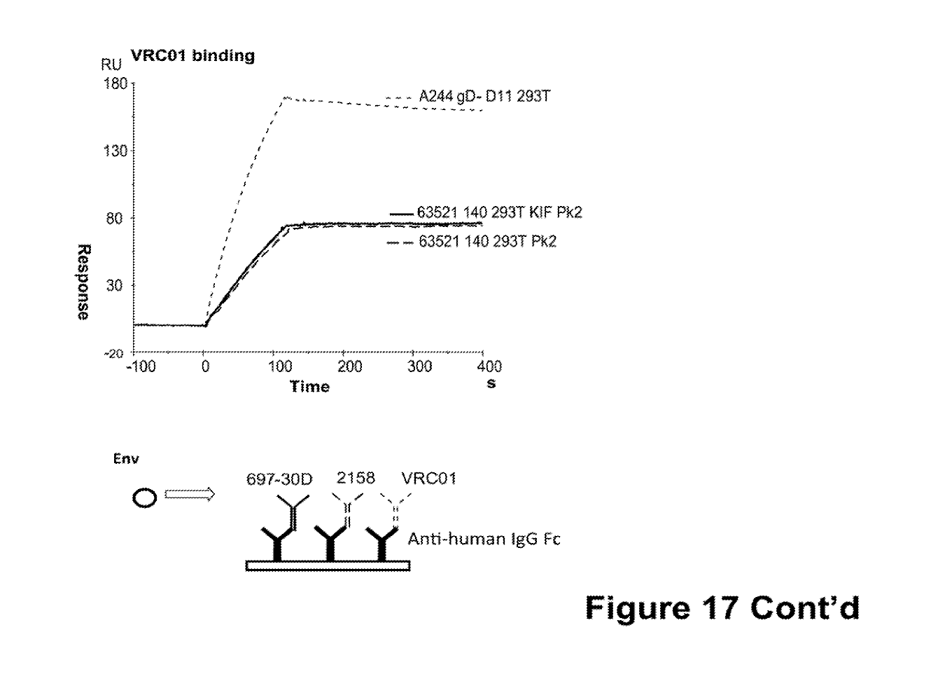

FIG. 17 shows that the V1V2 mabs 697d and 2158 bind to all three Envs A244 Delta 11 gp120, and to KIF treated and non-treated 63521.B gp140. Similarly the CD4 BS antibody VRC01 also binds to all three Envs. Kifunensine treatment does not alter the binding of these three antibodies.

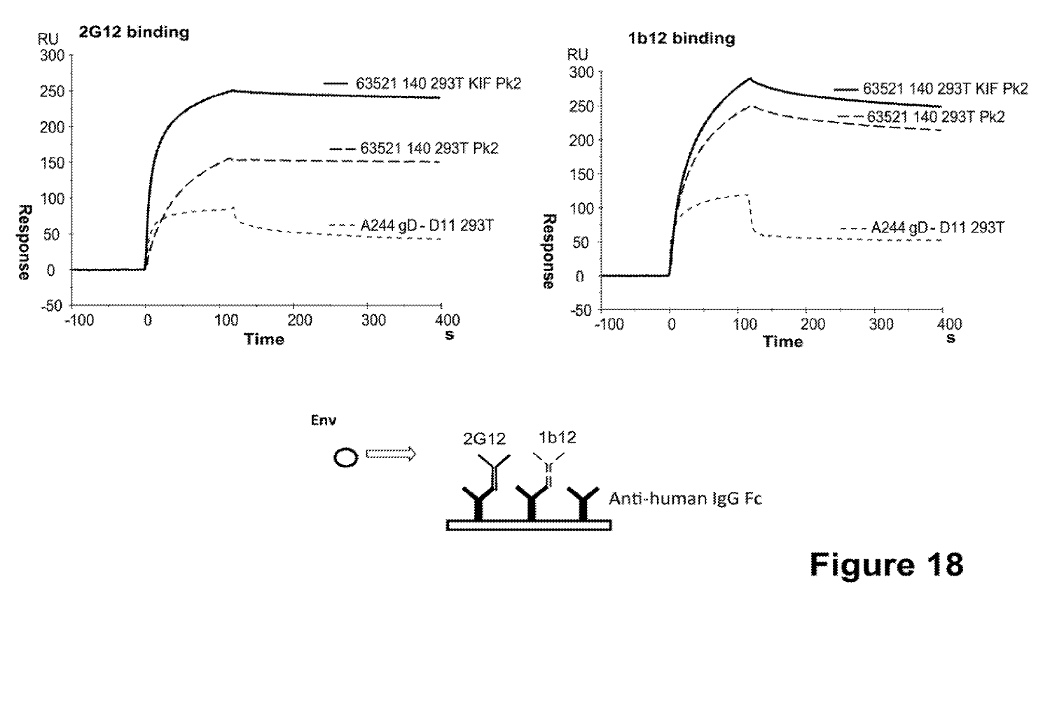

FIG. 18 shows however that kifunensine (KIF) treatment does upregulate the binding of 2G112 to 63521.B gp140C, while the binding of the CD4 binding site antibody 1b12 is minimally altered.

### Example 3

### TRL-7/8 and 9 Agonists Cooperate to Enhance HIV-1 Envelope Antibody Responses

The addition of toll-like receptor (TLR) agonists to boost vaccine responses has been suggested as one means of enhancing the response to HIV-1 immunogens (Karlsson et al, Nat. Rev. Microbiol. 6:143-155 (2008)). In order to determine whether the use of multiple TLR agonists could enhance the immunogenicity of a candidate HIV-1 envelope (Env) protein vaccine, a systematic comparison was undertaken in rhesus macaques of oil-in-water emulsions containing different combinations of TLR agonists formulated with a highly antigenic HIV-1 transmitted/founder envelope B.63521 gp140. It was found that a combination of TLR-7/8 and TLR-9 agonists optimally enhanced primate responses to HIV-1 Env. This enhanced response was associated with elevated levels of the chemokine CXCL 10 (IP-10) in plasma.

### EXPERIMENTAL DETAILS

### Adjuvant Production

The base adjuvant Span85/Tween80/squalene (STS) was prepared by mixing Span85, Tween 80, and squalene (Sigma-Aldrich, St. Louis, Mo.; catalog #s 85549, P8192, and 53626, respectively) at 0.5%, 0.5%, and 5% v/v, respectively, in IX phosphate buffered saline (PBS) (Gibco, Grand Island, N.Y.) (Ott et al, Vaccine 13:1557-1562 (1995)). For adjuvant combinations containing TLR agonists, 0.2 mg/mL lipid A (Avanti Polar Lipids, Alabaster, Ala.; catalog #699200P), 6.67 mg/mL CpG oligodeoxynucleotides (oCpGs; The Midland Certified Reagent Co., Midland, Tex.; catalog #ODN10103), and 1 mg/mL R848 (InvivoGen, San Diego, Calif.; catalog #Tlrl-r848-5) were added as shown in Table 2. In all cases, adjuvant mixtures were homogenized for 5 minutes at room temperature, using an OMNI International homogenizer using plastic soft tissue tips (Kennesaw, Ga.). Following initial homogenization, the adjuvant mixtures were further homogenized using a Microfluidizer model M-110S (Microfluidics Corp, Newton, Mass.). The cooling coil was kept on ice and the processor was primed three times with 8 mL of homogenized STS mixture, then each adjuvant mixture was pumped through the instrument at 14,000 psi, making 5 passes prior to collection of the final product. Stable emulsions were stored at room temperature prior to use.

TABLE 2
Adjuvant compositions.

| | TLR Agonists* | | |
|---|---|---|---|
| Adjuvant | Lipid A | oCpGs | R848 |
| STS | —^{†} | — | — |
| STS + LA | X | — | — |
| STS + oCpG | — | X | — |
| STS + R848 | — | — | X |
| STS + LA + oCpG | X | X | — |
| STS + LA + R848 | X | — | X |
| STS + oCpG + R848 | — | X | X |

*TLR agonists incorporated at 0.2 mg/mL for lipid A, 6.67 mg/mL for oCpGs, and 1 mg/mL for R848.
^{†}— = absent from formulation, X = present in formulation.

HIV-1 Envelope Proteins and V1V2 Reagents.

Envelope glycoproteins were produced as described for gp140 B.63521 (Tomaras et al, J. Virol. 82:12449-12463 (2008)), group M consensus gp140 ConS (Liao et al, Virology 353:268-282 (2006)), gp120 B.JRFL (Tomaras et al, J. Virol. 82:12449-12463 (2008)), gp120 E.A244gD+Δ11 (Alam et al, J. Virol. 87:1554-1568 (2013)), and E.A244gDneg (Alam et al, J. Virol. 87:1554-1568 (2013)). HIV-1 Env variable loop 1-variable loop 2 (V1V2) constructs for the detection of V1V2-specific antibodies were produced as described for A.Q23_V1V2, AE.A244_V1V2, and C.1086_V1V2 (Liao et al, Immunity 38:176-186 (2013)). In addition, constructs using murine leukemia virus (MLV) gp70 as a scaffold were prepared as described (Pinter et al, Vaccine 16:1803-1811 (1998)); the gp70 constructs included gp70_B.CaseA2_V1/V2 and MLV gp70 carrier protein without V1V2 sequence as a negative control.

Animal Studies.

Thirty-three adult rhesus monkeys (Macaca mulatta) were used in this study. All animals were housed at BioQual (Rockville, Md.) and maintained in accordance with the Association for Accreditation of Laboratory Animal Care guidelines at the National Institutes of Health. Twenty-one animals were immunized intramuscularly with gp140 B.63521 at 100 g/animal/immunization time point; each animal received 1 mL total injection volume divided into four sites. The final immunization cocktail contained 15% of adjuvant (Table 2), 0.1 mg/mL gp140 B.63521, with the remaining volume being sterile saline. Three animals per group were immunized for each of the 7 adjuvant formulations (Table 2); for this part of the study peripheral blood was obtained prior to study initiation, on each immunization day, and two weeks after each immunization.

To assess for adjuvant effect alone, 12 animals were immunized intramuscularly with adjuvant formulations in the absence of immunogen; these animals received the same total injection volume as those in the prior group. Three animals per group were used for this experiment that compared STS, STS+oCpG, STS+R848, and STS+oCpG+R848. For this part of the study, peripheral blood was obtained immediately prior to immunization and at 6 hours, 24 hours, 7 days, and 14 days after adjuvant administration.

Isolation of Plasma and Peripheral Blood Mononuclear Cells (PBM).

EDTA anti-coagulated blood from immunized monkeys was centrifuged over Ficoll (Ficoll-Paque) and plasma and PBMC layers were collected in separate tubes. PBMC were washed in IX PBS containing 2% FBS. Prior to use, plasma was aliquoted and stored at −80° C.; PBMC were cryopreserved in freezing media (10% dimethylsulfoxide/90% fetal bovine serum) and stored in the vapor phase of liquid nitrogen.

Antibody Characterization by ELISA.

Plasma samples were studied for reactivity to HIV-1 Env protein antigens and V1V2 constructs by ELISA as described (Ma et al, PLoS Pathog. 7:e1002200 (2011)). Blocking assays were performed as described (Tomaras et al, J. Virol. 82:12449-12463 (2008)) modified to use rhesus detection reagents (Ma et al, PLoS Pathog. 7:e1002200 (2011)). Plasma titers were determined using an initial 1:25 dilution (for Env reagents) or 1:30 (for V1V2 reagents) followed by a 3-fold dilution series; background for each analyte was set as the average of the final plasma. Endpoint titers were calculated by applying 4-parameter logistic regression to the binding data using the drc package in R (Ritz and Streibig, Bioassay Analysis Using R. Journal of Statistical Software 15:1-22 (2005)); endpoint was defined as OD=(3×background) for Env reagents and OD=(4×background) for V1V2 reagents.

Neutralization Assay in TZM-bl Cells.

Neutralizing antibody assays in TZM-bl cells were performed as described (Montefiori, Curr. Protoc. Immunol. Chapter 12:Unit 12.11 (2005)). Plasma samples were tested starting at a 1:20 dilution for the final concentration and titered using serial threefold dilutions. Pseudoviruses were added to the plasma dilutions at a predetermined titer to produce measurable infection and incubated for 1 h. TZM-bl cells were added and incubated for 48 h before lysis, after which supernatant was measured for firefly luciferase activity by a luminometer. The data were calculated as a reduction in luminescence compared with control wells and reported as plasma dilution IC₅₀ (Montefiori, Curr. Protoc. Immunol. Chapter 12:Unit 12.11 (2005)). All Env-pseudotyped viruses were prepared in 293T cells and titrated in TZM-bl cells as described (Montefiori, Curr. Protoc. Immunol. Chapter 12:Unit 12.11 (2005)).

Antibody-Dependent Cell-Mediated Cytotoxicity (ADCC) Assay.

ADCC assays were performed with plasma using HIV-1 A1953.B infected CEM.NKR_{CCR5} cells as described (Pollara et al, Cytometry A 79:603-612 (2011)).

Cytokine and Chemokine Assays.

Plasma from the second monkey group was assayed for the presence of cytokines/chemokines using a cytokine monkey magnetic 29-plex panel (Life Technologies, Frederick, Md.) and was performed per the manufacturer's instructions. Biomarker profiling was performed in the Duke Human Vaccine Institute Immune Reconstitution & Biomarker Analysis Shared Resource Facility (Durham, N.C.) under the direction of Dr. Gregory D. Sempowski. Plasma samples were also tested for interferon-α by capture ELISA per the manufacturer's instructions (Mabtech, Mariemont, Ohio).

Statistical analysis. Statistical tests were performed in SAS v9.2 (SAS Institute, Cary, N.C.). Comparisons of pre-planned contrasts for multiple groups were performed using multiple degree of freedom F-tests using PROC GLM in SAS with subsequent pairwise comparisons. When multiple comparisons were performed, p-values were corrected using the false discovery rate method (Benjamini and Hochberg, F R Statist. Soc. B 57:289-300 (1995)). The statistical test used is noted when p-values are presented. Graphs of the data were created using GraphPad Prism (GraphPad Software, La Jolla, Calif.) with layout in Illustrator CS5 (Adobe, San Jose, Calif.).

### Results

Oil-in-Water Emulsion Adjuvants Combined with Env Immunogens Elicit HIV-1 Env-Reactive Antibodies.

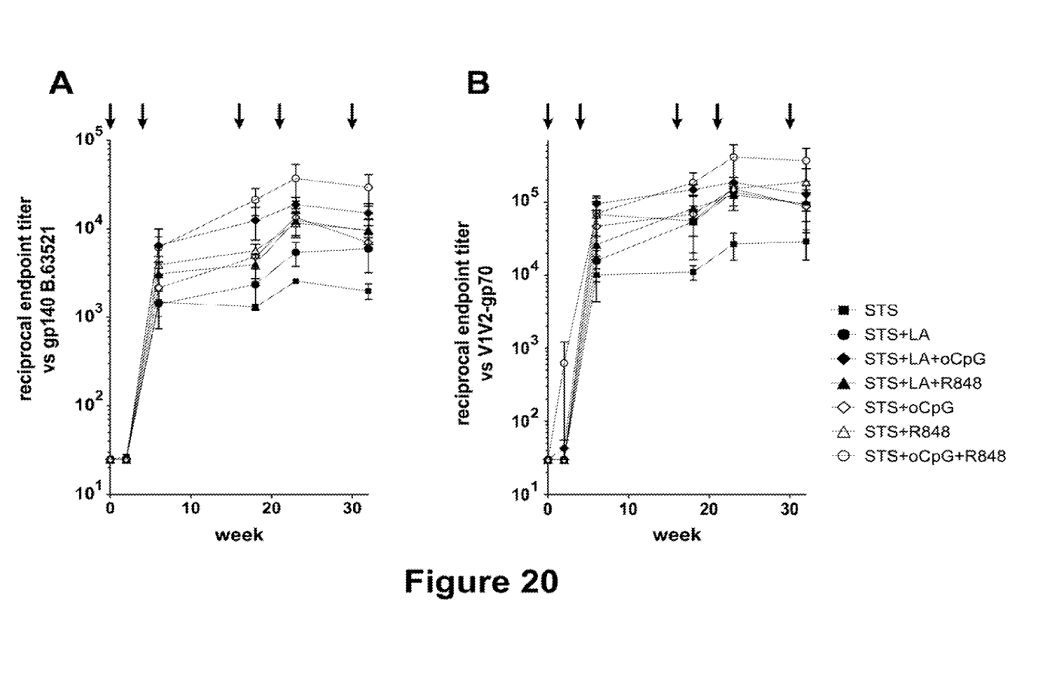

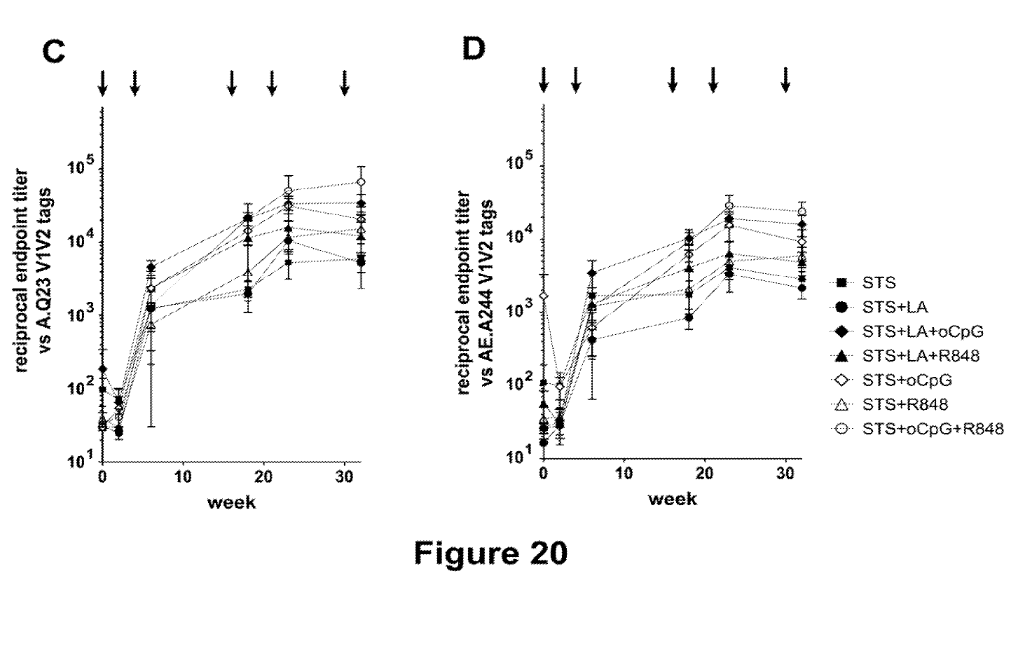

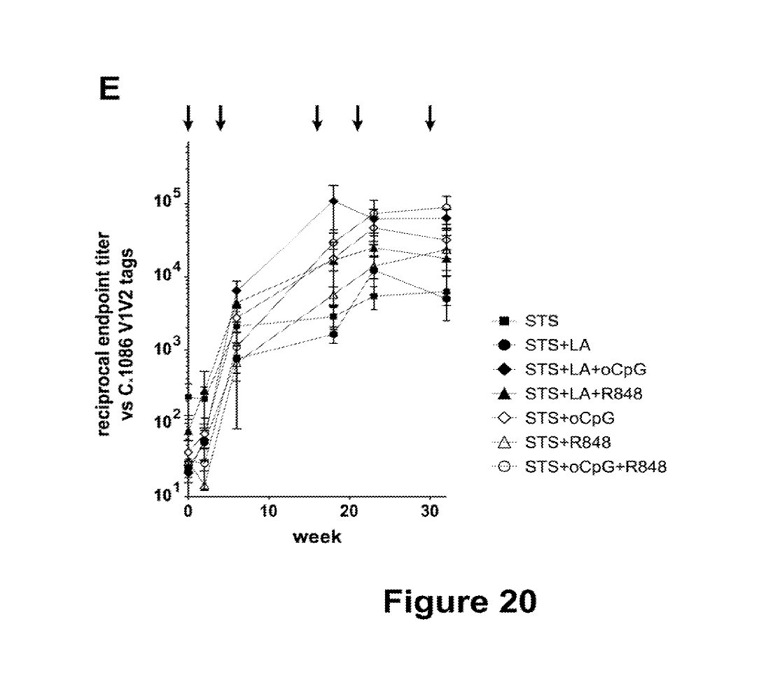

An assessment was first made of the ability of the different squalene-based adjuvant formulations (Table 2) to induce antibodies reactive with the transmitted/founder Env immunogen, gp140 B.63521. Env gp140 B.63521 is a highly antigenic protein that expresses sites for broadly neutralizing monoclonal antibodies (mAbs) directed against glycans, variable loop 1-variable loop 2 (V1V2), the CD4 binding site (CD4bs), and the membrane proximal external region (MPER). After two immunizations, all animals developed robust titers against gp140 B.63521 that remained elevated for the remainder of the study (FIG. 20A). After the fifth immunization, animals immunized with adjuvant STS had the lowest endpoint titer (1:1,905; 95% confidence interval [CI]1:728-1:4,989) while those animals immunized with STS+oCpG+R848 had the highest endpoint titer (1:25,704; 95% CI 1:5,420-1:121,899; t-test p=0.004).

TLR-Agonists Enhance Epitope-Specific HIV-1 Env Reactive Antibody Levels.

The plasma samples were further assessed for the presence of epitope-specific antibodies through direct binding assays. The RV144 ALVAC HIV-1/AIDSVAX® B/E vaccine trial demonstrated 31.2% vaccine efficacy (Rerks-Ngarm et al, N. Engl. J. Med. 361:2209-2220 (2009)), and the immune correlates analysis showed a direct correlation between antibodies directed against V1V2 and a decreased risk of infection (Haynes et al, N. Engl. J. Med. 366:1275-1286 (2012)). All rhesus macaque groups in the current study developed antibodies that bound to B.CaseA2 V1V2-gp70, the same protein used in the immune correlates case-control study (Haynes et al, N. Engl. J. Med. 366:1275-1286 (2012)) (FIG. 20B). After the fifth immunization, adjuvant STS again elicited the lowest endpoint titer (1:19,890; 95% CI 1:912-1:434,011) while STS+oCpG+R848 elicited the highest titer (1:298,498; 95% CI 1:44,722-1:1,992,000). An analysis was made for the presence of V1V2 cross-clade reactivity and a similar trend was found over titers against clade A, CRF01_AE, and C V1V2 protein constructs (FIGS. 20C, 20D, and 20E, respectively).

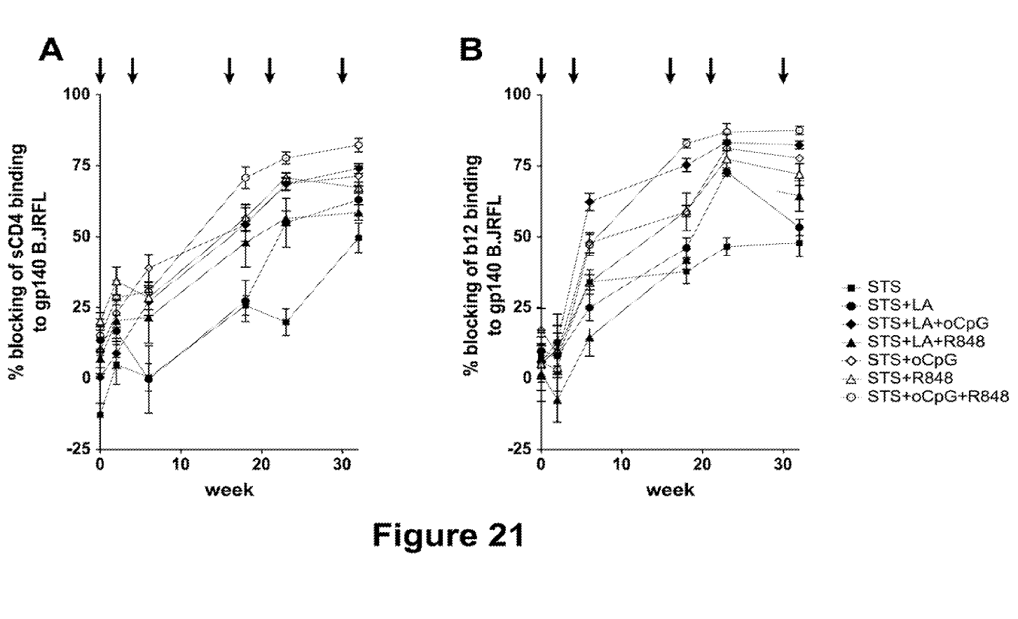

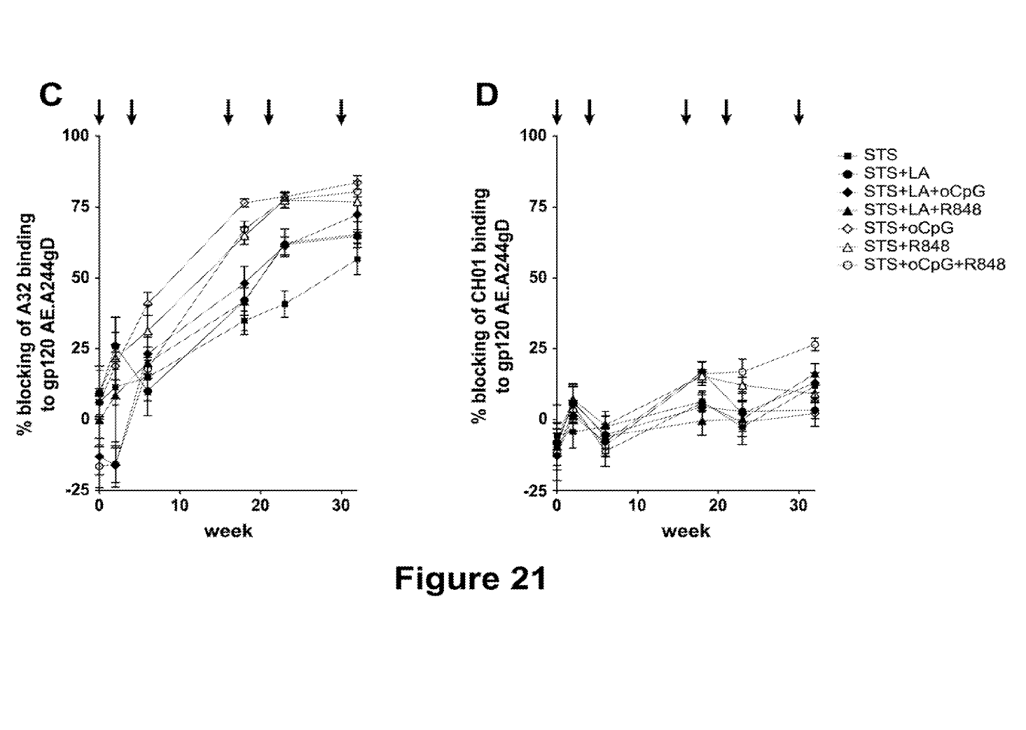

A search was then made for the presence of antibodies against other known specificities through the use of assays of plasma competition with mAbs of known specificity or soluble CD4 (sCD4). All adjuvant combinations were able to elicit antibodies that blocked the binding of sCD4 and mAb b12 to gp140 B.JRFL (FIGS. 21A and 21B, respectively). Similar to the pattern observed with overall Env binding, after five immunizations antibodies were lowest for STS and highest for STS+oCpG+R848, both for those that blocked sCD4 binding (blocking of 50% and 82%, respectively; FIG. 21A) and those that blocked CD4bs mAb b12 binding (blocking of 48% and 88%, respectively; FIG. 21B). Blocking of ADCC-mediating mAb A32 showed a different pattern; after five immunizations, STS elicited 57% blocking while STS+R848 was slightly higher than STS+oCpG+R848 (84% vs. 81%, respectively; FIG. 21C). No adjuvant combination elicited high level blocking of V1V2-binding broadly neutralizing mAb CH01; however, STS+oCpG+R848 did elicit low level blocking after five immunizations (27%; FIG. 21D).

Combined TLR Agonists Elicit Higher Titers of Neutralizing and ADCC-Mediating Antibodies.

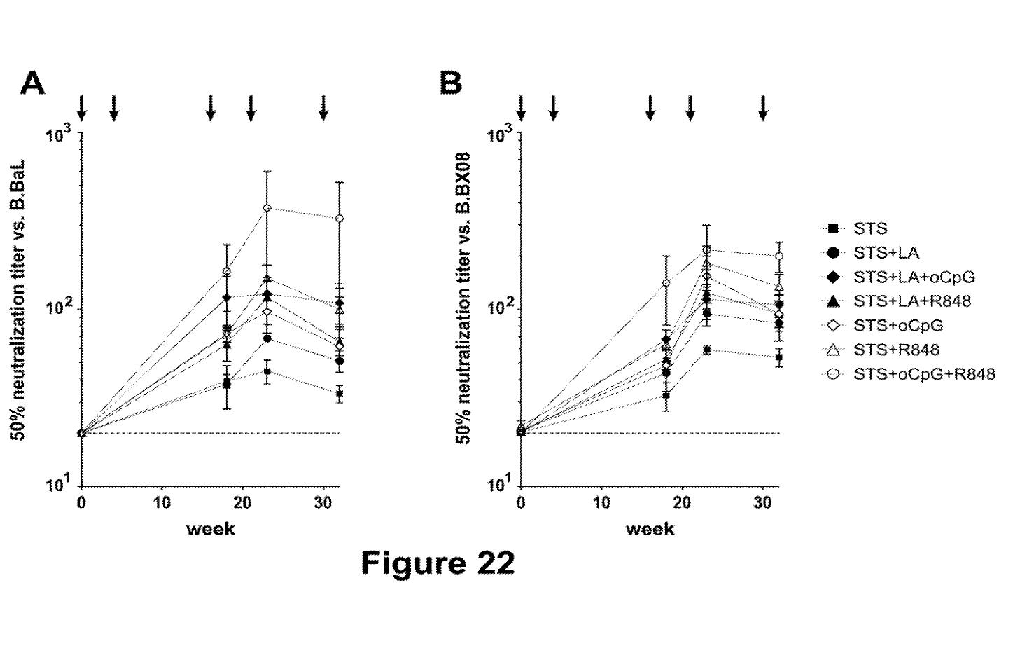

The ability of vaccine-elicited antibodies to neutralize HIV-1 in the TZM-bl pseudovirus neutralization assay was tested next. Similar to what was observed for binding antibody titers, the 50% neutralization titers against B.BaL and B.BX08 were lowest for STS alone and highest for STS+oCpG+R848 (FIG. 22). The largest difference in neutralization activity was observed after the fourth immunization and titers were found to be slightly lower after the fifth immunization (FIG. 22). The neutralization titer against B.BaL elicited by STS was 1:45 while that elicited by STS+oCpG+R848 was 1:374 (t-test p<0.05); similarly, titers against B.BX08 elicited by these two adjuvant combinations after four immunizations were 1:59 and 1:216, respectively (t-test p<0.05).

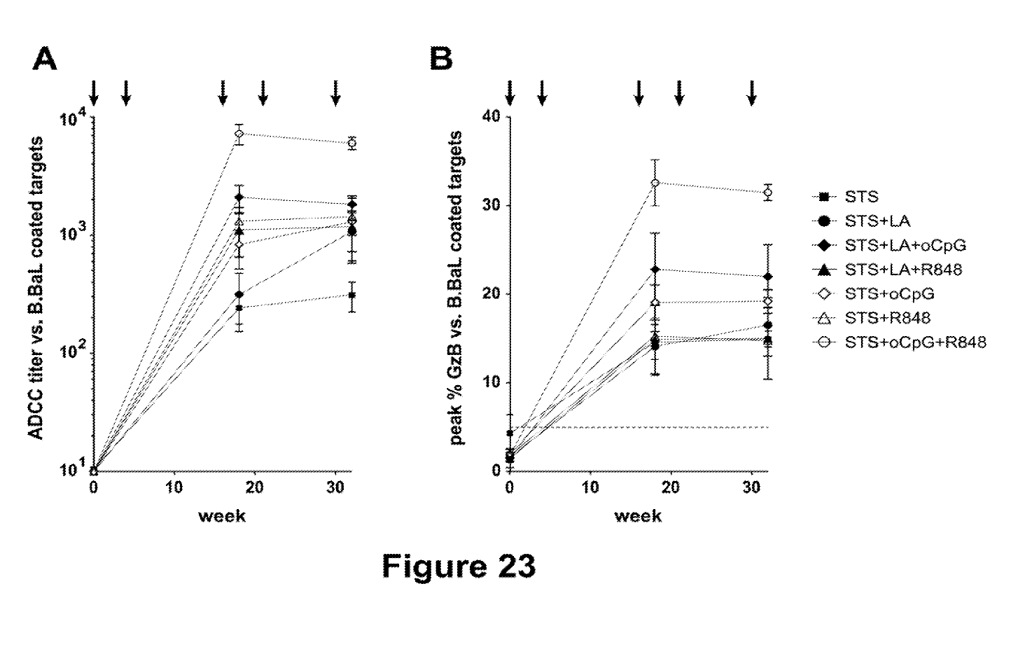

Next, the ability of vaccine-elicited antibodies to mediate ADCC against B.BaL coated target cells was tested (FIG. 23). After five intramuscular immunizations, STS elicited the lowest endpoint ADCC titer (1:2,317, 95% CI 1:579-1:9,268) while STS+oCpG+R848 elicited the highest titer (1:47,753, 95% CI 1:27,227-1:83,946; t-test p=0.001; FIG. 23A). Peak activity in the ADCC assay displayed a similar pattern, with peak activity elicited by STS at 14.9%±0.9% versus that elicited by STS+oCpG+R848 at 31.5%±0.9% (t-test p=0.0002; FIG. 23B). ADCC activity elicited by STS+oCpG+R848 was markedly higher than that elicited by any other adjuvant tested; the next highest group after five immunizations was STS+LA+oCpG, which elicited an endpoint titer of 1:18,290 and peak activity of 22.0% (FIG. 23).

Formulation of TLR7/8 and TLR9 Selectively Results in Elevation of Plasma CXCL10 (IP-10).

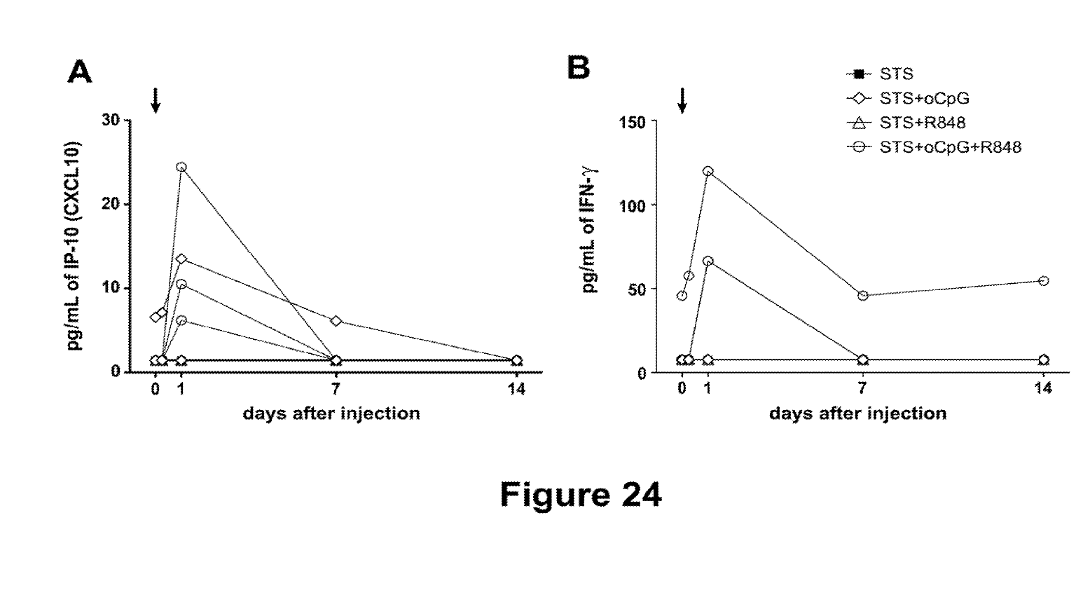

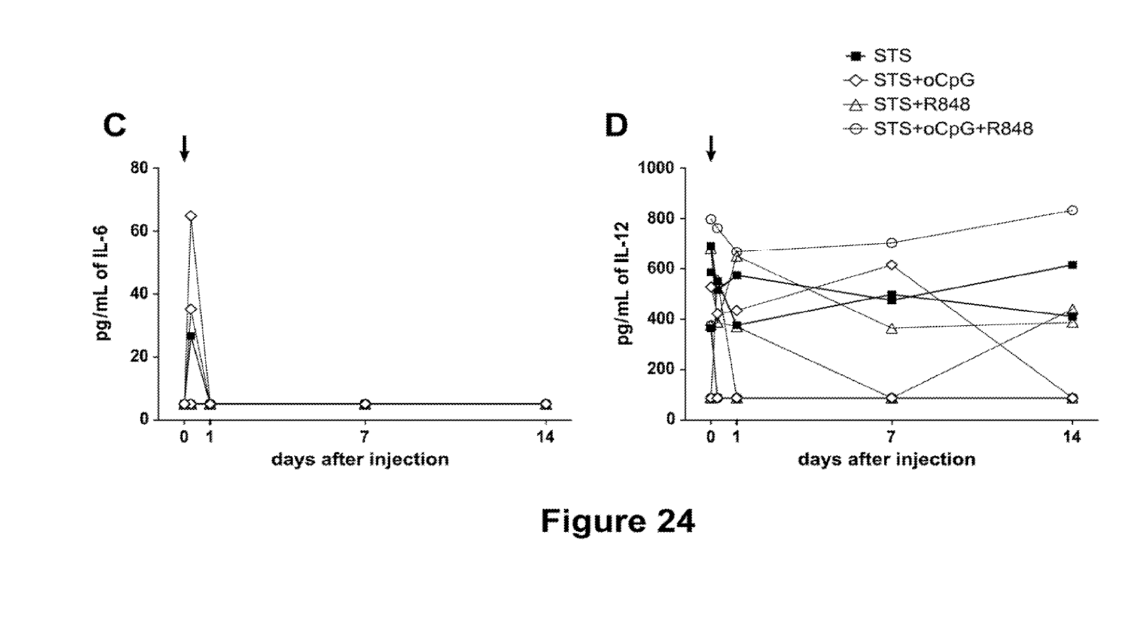

A determination was next made as to whether TLR agonist combinations could elicit cytokines and chemokines that correlate with the observed differences in induced antibody levels. Using a separate group of naïve rhesus macaques, immunization was effected with oil-in-water emulsions containing TLR agonists. Plasma samples were obtained after 6 hours, 24 hours, one week, and two weeks; and tested for the presence of 30 cytokines/chemokines. Across all five time points, no detectable changes were found for eleven markers (interferon [IFN]-α, interleukin [IL]-4, IL-5, IL-10, IL-5, IL-17, granulocyte-monocyte colony stimulating factor [GM-CSF], granulocyte colony stimulating factor [G-CSF], macrophage inflammatory protein [MIP]-1α, MIP-1β, vascular endothelial growth factor [data not shown]). For 16 markers, detectable changes were observed across different time points, but without a discernable pattern related to immunization; representative data are for IL-12 shown (FIG. 24D), and similar non-specific patterns were observed for 15 other markers (IL-1 receptor α, IL-β, IL-2, IL-8, fibroblast growth factor basic, monocyte chemotactic protein [MCP]-1, eotaxin, RANTES, epidermal growth factor, hepatocyte growth factor, chemokine (C-C motif) ligand [CCL]-22, chemokine (C-X-C motif) ligand [CXCL]-9, CXCL-11, macrophage migration inhibitory factor [MIF], tumor necrosis factor α [data not shown]).

A transient elevation of IFN-γ was observed in 2/3 animals immunized with STS+oCpG+R848; the elevation peaked at 24 hours and had returned to baseline by the one week time point (FIG. 24B). Similarly, a transient elevation of IL-6 was observed in 2/3 animals immunized with STS+oCpG and in 1/3 animals immunized with STS alone, with the peak occurring at 6 hours and returning to baseline by 24 hours (FIG. 24C). These elevations were not observed in any of the other immunized animals (FIGS. 24B and 24C).

When CXCL10 (interferon-γ-induced protein [IP]-10) was measured, it was found that 3/3 animals immunized with STS+oCpG+R848 had elevated levels that peaked 24 hours after immunization and that returned to baseline by the one week time point (FIG. 24A). Only one other animal, immunized with STS+oCpG, had elevated levels of CXCL10; this animal had higher levels at baseline that then returned to baseline by the one week time point (FIG. 24A).

Summarizing, in this study, it has been demonstrated that a combination of a TLR-9 agonist (type B oCpG [ODN10103]) with a TLR-7/8 agonist (R848) formulated in an oil-in-water emulsion with transmitted/founder Env gp140 B.63521 resulted in significantly higher levels of ADCC and tier 1 neutralizing antibodies compared to other TLR agonist combinations. Adjuvants stimulate immune responses through triggering of host defense pathways designed to recognize damage or threats. By combining agonists for different molecular pattern recognition pathways, an adjuvant can trigger signaling events that activate both immediate inflammatory responses and later adaptive T and B cell anti-pathogen responses (Schenten and Medzhitov, Adv. Immunol. 109:87-124 (2011), Olive, Expert Rev. Vaccines 11:237-256 (2012)). Using a combination of stimuli to selectively trigger the immune system using an adjuvant formulation will be critical for enhancing vaccine responses against HIV-1 Env immunogens.

There is a global need for an effective vaccine against HIV-1 (Kim et al, Curr. Opin. HIV AIDS 5:428-434 (2010)), but to date only one of the four HIV-1 vaccine efficacy trials in humans has shown any degree of protection from infection (Rerks-Ngarm et al, N. Engl. J. Med. 361:2209-2220 (2009), Fitzgerald et al, J. Infect. Dis. 203:765-772 (2011), Buchbinder et al, Lancet 372:1881-1893 (2008), Pitisuttithum et al, J. Infect. Dis. 194:1661-1671 (2006), Flynn et al, J. Infect. Dis. 191:654-665 (2005)). Although the estimated vaccine efficacy afforded by the RV144 ALVAC HIV-1/AIDSVAX® B/E vaccine regimen was modest and short-lived (Rerks-Ngarm et al, N. Engl. J. Med. 361:2209-2220 (2009)), a correlates of risk analysis showed that higher levels of IgG antibodies against V1V2 directly correlated with decreased risk of infection (Haynes et al, N. Engl. J. Med. 366:1275-1286 (2012)). Moreover, it has recently been shown that RV144 vaccine-elicited antibodies directed against specific epitopes in the V1V2 loops can mediate ADCC (Bonsignori et al, J. Virol. 86:11521-11532 (2012)) and neutralize some isolates of HIV-1 (Liao et al, Immunity 38:176-186 (2013), Montefiori et al, J. Infect. Dis. 206:431-441 (2012)). A major problem with the alum-based vaccine used in RV144 was that antibody responses declined over the first year following completion of the vaccine regimen, such that the estimated vaccine efficacy at one year was 60.5% (Robb et al, Lancet Infect. Dis. 12:531-537 (2012)) and at three years was 31.2% (Rerks-Ngarm et al, N. Engl. J. Med. 361:2209-2220 (2009)). While much work remains to develop novel immunogens that can extend these results, the parallel development of adjuvants that enhance desirable responses is critically important.

One desirable feature in an adjuvant formulation is that it not perturb the antigenicity of the vaccine insert. For this reason it was important that the protein immunogen, transmitted/founder Env gp140 B.63521, retained antigenicity to a panel of mAbs representing targets of HIV-1 vaccine development.

To date, regulatory authorities in the United States have only licensed two adjuvants for human use: alum which is used in a number of vaccines (Baylor et al, Vaccine 20(Suppl. 3):S18-23 (2002)), and a lipid-based adjuvant system formulated with a human papillomavirus vaccine (Centers for Disease Control and Prevention CDC, FDA lincensure of bivalent human papillomavirus vaccine (HPV2, Cervarix) for use in females and updated HPV vaccination recommendations from the Advisory Committee on Immunization Practices (ACIP), MMWR Morb. Mortal. Wkly. Rep. 59:626-629 (2010)). However, even though they were not added to the vaccine formulation, it has been shown that the presence of “hidden” TLR agonists enhances the immunogenicity of FDA-approved vaccines directed against Streptococcus pneumoniae (Sen et al, J. Immunol. 175:3084-3091 (2005)). In addition, live attenuated vaccines trigger TLR pathways during the time of abortive infection that induces long-lasting immunity (Pulendran, Nat. Rev. Immunol. 9:741-747 (2009)). Thus, there is precedent for the use of TLR agonists in vaccines, and the FDA has issued guidance on what would be needed to license new adjuvants in the context of influenza vaccination (Guidance for Industry: Clinical Data Needed to support the Licensure of Pandemic Influenza Vaccinesfda.gov., Food and Drug Administration (2007)).

Although both TLR7 and TLR9 appear to converge on the same signaling pathway, enhancement of vaccine response was observed using a combination of ligands for these two receptors. TLR 7 (Hemmi et al, Nat. Immunol. 3:196-200 (2002)) and TLR 9 (Hemmi et al, J. Immunoo. 170:3059-3064 (2003)) both act through MyD88, and so the increase in activity found through the use of this combination was not expected. The pathogen ligands for these two TLRs differ (single stranded RNA for TLR7/8 and CpG DNA for TLR9 (Wickelgren, Science 312(5771):184-187 (2006)), thus differences in their downstream effects might be expected, and the present data suggest that combined triggering can lead to desirable responses. There is evidence that other combinations of TLR agonists can combine to enhance vaccine response, such as combinations of TLR3 and TLR4 with TLR7, TLR8, and TLR9 (Napolitani et al, Nat. Immunol. 6:769-776 (2005)). Since it is possible to incorporate multiple TLR agonists in liposomal particles as an effective adjuvant system, as has been reported for the combination of TLR7 and TLR9 agonists in activating polyreactive B cells, it may be possible to use multiple delivery vehicles to administer combinations of TLR agonists that can enhance vaccine responses.

It was found that there was a transient elevation of CXCL10 (IP-10) following vaccination with combined TLR7/8 and TLR9 agonists. These agonists have been shown to stimulate IP-10 secretion in rhesus macaques when administered individually (Kwissa et al, Blood 119:2044-2055 (2012)). Furthermore, secretion of IP-10 triggered by TLR agonists has been shown to cause regulatory dendritic cells to recruit Th1 cells and to then inhibit their proliferation (Qian et al, Blood 109:3308-3315 (2007)). Given the role of Th1 cells in promoting cellular immunity over humoral immunity (Zygmunt and Veldhoen, Adv. Immunol. 109:159-196 (2011)), inhibition of this helper T cell subset may explain why IP-10 elevation correlated with enhanced antibody responses.

In conclusion, it has been shown in the study described above that inclusion of TLR-7/8 and TLR-9 agonists in a squalene-based oil-in-water emulsion improves induction of HIV-1 antibodies. Such an adjuvant regimen does not perturb the antigenicity of recombinant HIV-1 Envs, and should be a powerful adjuvant formulation to use with highly antigenic Envs that can induce high titers of potentially protective antibodies.

All documents and other information sources cited above are hereby incorporated in their entirety by reference.

1.-15. (canceled)

16. An immunogenic composition comprising a TLR7 or a TLR7/8 agonist and a TLR-9 agonist and an immunogen.

17. The composition of claim 16 wherein the immunogen is HIV-1 Env.

18. The composition of claim 17, wherein the HIV-1 Env immunogen is the 63521 clade B transmitted/founder envelope.

19. The composition of claim 17, wherein the HIV-1 Env immunogen has high-mannose glycan residues on the surface thereof.

20. The composition of claim 19 wherein the highmannose glycan residues are man(4), man(5), man(7) or man(8) residues.

21. The composition of claim 16 further comprising a squalene-based oil-in-water emulsion.

22. The composition of claim 17 further comprising a squalene-based oil-in-water emulsion.

23. The composition of claim 16 further comprising phosphate buffered saline, squalene, polysorbate 80 and sorbitan trioleate.

24. The composition of claim 17 further comprising phosphate buffered saline, squalene, polysorbate 80 and sorbitan trioleate.

25. A method/use of the composition of claim 16 to induce an antibody response to an immunogen in a subject comprising: administering to the subject the composition of claim 16 in an amount sufficient to effect the stimulation.
